(19) United States
(12) Patent Application Publication
(10) Pub. No.: US 2024/0285680 A1
(43) Pub. Date: Aug. 29, 2024

(54) METHOD OF TREATING ALZHEIMER'S DISEASE

(71) Applicant: Vector Vitale IP LLC, North Miami Beach, FL (US)

(72) Inventors: Peter Y. NOVAK, Sunny Isles Beach, FL (US); Maxim V. TEMNIKOV, Miami, FL (US)

(73) Assignee: Vector Vitale IP LLC, North Miami Beach, FL (US)

(21) Appl. No.: 18/581,659

(22) Filed: Feb. 20, 2024

Related U.S. Application Data
(63) Continuation of application No. 17/214,377, filed on Mar. 26, 2021, now abandoned.

(51) Int. Cl. A61K 33/30; A61K 9/00; A61P 25/28

(52) U.S. Cl. CPC A61K 33/30; A61K 9/0019; A61K 9/0053; A61P 25/28

(57) ABSTRACT

A method of treating or preventing Alzheimer's disease comprising administering a pharmaceutical composition comprising ⁶⁴Znₑ or a salt thereof, at a therapeutically effective or a prophylactically effective dose for treating or preventing the disease.

### TECHNICAL FIELD

This disclosure relates to therapy for Alzheimer's disease.

### BACKGROUND

Alzheimer's disease (“AD”) is the most prevalent form of dementia, affecting more than 37 million people worldwide. Mount C, Downton C., Nature Medicine 2006; 12(7):780-784; Wimo A et al., Alzheimer's and Dementia 2010; 6(2):98-103. In the modern society, AD is of great medical and social concern, considering that the incidence of the illness grows and the prospect of an aging population will result in rising social and economic demands.

AD is characterized by the presence of extracellular amyloid plaques and intracellular neurofibrillary tangles within the afflicted brain, which cause neuronal loss in the neocortex, hippocampus, and basal forebrain, leading to progressive cognitive and behavioral decline. Watt N T et al., Int J Alzheimers Dis. 2010; 2011:971021 Published 2010 Dec. 20, doi.10.4061/2011/971021.

There is no cure for AD.

### SUMMARY

In one aspect, this disclosure provides a method of treating or preventing AD in a patient, comprising administering to said patient a pharmaceutical composition comprising zinc, at a therapeutically effective or a prophylactically effective dose for treating or prevent the disease.

In some embodiments, the composition comprises ⁶⁴Zn-enriched zinc (the term “⁶⁴Znₑ” is used herein to refer to ⁶⁴Zn-enriched zinc).

In some embodiments, the ⁶⁴Zn-enriched zinc is in the form of a ⁶⁴Znₑ compound or a ⁶⁴Znₑ salt. In certain embodiments, the disclosed compositions contain zinc that is at least 80% ⁶⁴Znₑ, at least 90% ⁶⁴Znₑ, at least 95% ⁶⁴Znₑ, or at least 99% ⁶⁴Znₑ, for example, zinc that is 80% ⁶⁴Znₑ, 85% ⁶⁴Znₑ, 90% ⁶⁴Znₑ, 95% ⁶⁴Znₑ, 99% ⁶⁴Znₑ, or 99.9% ⁶⁴Znₑ.

Numerous other aspects are provided in accordance with these and other aspects of the invention. Other features and aspects of the present invention will become more fully apparent from the following detailed description and the appended claims.

### BRIEF DESCRIPTION OF THE DRAWINGS

FIG. 1 is a graphical presentation of the experimental design in the study of therapeutic effects of ⁶⁴Zn-asp on behavioral functions of experimental models of Alzheimer's Disease.

FIG. 2 shows the Barnes maze.

FIG. 3 shows the effects of ⁶⁴Zn-asp on the body weight in rat models of Aβ1-40-induced Alzheimer's disease. The data are presented as a percentage ratio of the final body weight of an animal (on the day of autopsy) to its body weight before mimicking Alzheimer's disease, which was taken as 100%.

FIG. 4A and FIG. 4B show the effects of ⁶⁴Zn-asp on the eating (food intake) (FIG. 4B) and drinking (water intake) (FIG. 4A) behavior in rat models of Aβ1-40-induced Alzheimer's disease. The animals were placed in individual cages and the amounts of food and water they consumed were measured daily for each rat, starting from the 18th day (8 days after surgery) and until the end of the experiment (37th day). The data were first averaged into 1 rat per day within a group, and then into 1 rat per day for the entire period of observation.

FIG. 5A-FIG. 5D Immunohistochemical identification of tyrosine hydroxylase (TH) activity in the neurons in the hippocampus of intact rats (FIG. 5A), sham-operated rats (placebo) (FIG. 5B), rat models of Aβ1-40-induced Alzheimer's disease injected with H₂O (FIG. 5C) and rat models of Aβ1-40-induced Alzheimer's disease treated with 64Zn-asp (FIG. 5D). TH-positive staining (dark). Oc. 40, ob. 10.

FIG. 6A (before the operation) and FIG. 6B (after the operation) are graphs showing time spent by rat models of Aβ1-40-induced Alzheimer's disease for spatial learning in the Barnes maze. Data are presented as an average of 4 trials every 15 min/rat/day and an average within each group/day. M±SD

FIG. 7A (before the operation) and FIG. 7B (after the operation) show the effects of ⁶⁴Zn-asp on the spatial short-term and long-term memory efficiency (time spent to find an entrance to the “escape box”) and cognitive flexibility (time spent near the entrance to the “escape box”) 24 hours (5^{th} day after the 4-day training) and 5 days after training (9^{th} day after the 4-day training) in rat models of Aβ1-40-induced Alzheimer's. disease, M±SD

FIG. 8 shows therapeutic effects of ⁶⁴Zn-asp on the Aβ levels in the hippocampus of rat models of AD induced by the infusion of Aβ1-40 (n=5 in all groups). A is a relevant number of cells performing phagocytosis; B is phagocytic activity. *p<0.05 versus intact animals

FIG. 9 shows therapeutic effects of ⁶⁴Zn-asp on the tau protein levels in the hippocampus of rat models of AD induced by the infusion of Aβ1-40 (n=5 in all groups). A is a relevant number of cells performing phagocytosis; B is phagocytic activity. *p<0.05 versus intact animals

FIG. 10A and FIG. 10B show therapeutic effects of ⁶⁴Zn-asp on the microglial phagocytosis in rat models of AD induced by the infusion of Aβ1-40 (n=5 in all groups). FIG. 10A shows relevant number of cells performing phagocytosis; FIG. 10B shows phagocytic activity. *p<0.05 versus intact animals

FIG. 11 shows therapeutic effects of ⁶⁴Zn-asp on the oxidative metabolism in microglia in rat models of AD induced by the infusion of Aβ1-40 (n=5 in all groups). *p<0.05 versus intact animals; #≤0.05 versus control AD animal models

FIG. 12A and FIG. 12B show the expression of CD86 in microglia in 64Zn-asp treated rat models of Alzheimer's disease induced by the infusion of Aβ1-40 (n=5 in all groups). FIG. 12A is the number of expressing cells in the population analyzed; FIG. 12B is the level of expression. *p<0.05 versus intact animals; #≤0.05 versus control AD animal models

FIG. 13A and FIG. 13B show the expression of CD206 in microglia in ⁶⁴Zn-asp treated rat models of Alzheimer's disease induced by the infusion of Aβ1-40 (n=5 in all groups). FIG. 13A is the number of expressed cells in the population analyzed; FIG. 13B is the level of expression. *−p<0.05 versus intact animals; #−≤0.05 versus control AD animal models

FIG. 14 shows the effects of ⁶⁴Zn-asp on the levels of circulating leukocytes in rat models of Aβ1-40-induced AD, M±SD. Note: *p<0.05 vs. intact animals, #p<0.05 vs. untreated animal models of AD

FIG. 15A (number of cells performing phagocytosis, %) and FIG. 15B (phagocytosis index, GMean) show effects of ⁶⁴Zn-asp on phagocytic activity of circulating polymorphonuclear granulocytes in rat models of Aβ1-40-induced AD, M±SD Note: *p<0.05 vs. intact animals, #p<0.05 vs. untreated animal models of AD

FIG. 16A (number of cells performing phagocytosis, %) and FIG. 16B (phagocytosis index, GMean) show effects of ⁶⁴Zn-asp on phagocytic activity of circulating monocytes in rat models of Aβ1-40-induced AD, M±SD Note: *p<0.05 vs. intact animals, #p<0.05 vs. untreated animal models of AD

FIG. 17A and FIG. 17B show Effect of ⁶⁴Zn-asp on the oxidative metabolism in circulating granulocytes (FIG. 17A) and monocytes (FIG. 17B) in rat models of Aβ1-40-induced AD. Note: *p<0.05 vs. intact animals; ^{#}<0.05 vs. untreated AD animal models

FIG. 18A and FIG. 18B show the expression of CD86 in the population of circulating phagocytes in ⁶⁴Zn-asp treated rat models of Alzheimer's disease induced by the infusion of Aβ1-40 (n=5 in all groups). FIG. 18A is a number of expressing cells in the population analyzed; FIG. 18B is the level of expression. *p<0.05 versus intact animals; ^{#}≤0.05 versus control AD animal models

FIG. 19A and FIG. 19B show the expression of CD206 in the population of circulating phagocytes in ⁶⁴Zn-asp treated rat models of Alzheimer's disease induced by the infusion of Aβ1-40 (n=5 in all groups). FIG. 19A is the number of expressing cells in the population analyzed; FIG. 19B is the level of expression. *p<0.05 versus intact animals; ^{#}≤0.05 versus control AD animal models

FIG. 20 shows effects of ⁶⁴Zn-asp on the body weight in rat models of Aβ25-35-induced Alzheimer's disease. The data are presented as a percentage ratio of the final body weight of an animal (on the day of autopsy) to its body weight before mimicking Alzheimer's disease, which was taken as 100%.

FIG. 21A shows immunohistochemical identification of tyrosine hydroxylase activity in the neurons in the hippocampus of intact rats. FIG. 21B shows immunohistochemical identification of tyrosine hydroxylase activity in the neurons sham-operated rats (placebo). FIG. 21C shows immunohistochemical identification of tyrosine hydroxylase activity in the neurons rat models of Aβ25-35-induced Alzheimer's disease injected with H₂O. FIG. 21D shows immunohistochemical identification of tyrosine hydroxylase activity in the neurons and rat models of Aβ25-35-induced Alzheimer's disease treated with ⁶⁴Zn-asp. TH-positive staining (dark). Oc. 40, ob. 10.

FIG. 22A (before the operation) and FIG. 22B (after the operation) show time spent by rat models of Aβ25-35-induced Alzheimer's disease for spatial learning in the Barnes maze. Data are presented as an average of 4 trials every 15 min/rat/day and an average within each group/day. M±SD

FIG. 23A and FIG. 23B show therapeutic effects of ⁶⁴Zn-asp on the Aβ (A) and tau protein (B) levels in the hippocampus of rat models of AD induced by the infusion of Aβ25-35 (n=5 in all groups). FIG. 23A is the relevant number of cells performing phagocytosis; FIG. 23B is phagocytic activity. *p<0.05 versus intact animals

FIG. 24A and FIG. 24B display therapeutic effects of ⁶⁴Zn-asp on the microglial phagocytosis in rat models of AD induced by the infusion of Aβ25-35 (n=5 in all groups). FIG. 24A is the relevant number of cells performing phagocytosis; FIG. 24B is phagocytic activity. *p<0.05 versus intact animals; ^{#}p<0.05 versus control rat models of AD

FIG. 25 shows therapeutic effects of ⁶⁴Zn-asp on the oxidative metabolism in microglia in rat models of AD induced by the infusion of Aβ25-35 (n=5 in all groups). *p<0.05 versus intact animals; ^{#}≤0.05 versus control AD animal models

FIG. 26A and FIG. 26B show the expression of CD86 in microglia in ⁶⁴Zn-asp treated rat models of Alzheimer's disease induced by the infusion of Aβ25-35 (n=5 in all groups). FIG. 26A is the number of expressing cells in the population analyzed; FIG. 26B is the level of expression. *p<0.05 versus intact animals; ^{#}≤0.05 versus control AD animal models

FIG. 27A and FIG. 27B show the expression of CD206 in microglia in ⁶⁴Zn-asp treated rat models of Alzheimer's disease induced by the infusion of Aβ25-35 (n=5 in all groups). FIG. 27A is the number of expressing cells in the population analyzed; FIG. 27B is the level of expression. *p<0.05 versus intact animals; ^{#}≤0.05 versus control AD animal models

FIG. 28 shows the effects of ⁶⁴Zn-asp on the levels of circulating leukocytes in rat models of Aβ25-35-induced AD, M±SD. Note: *p<0.05 vs. intact animals, #p<0.05 vs. untreated animal models of AD

FIG. 29A and FIG. 29B show therapeutic effects of ⁶⁴Zn-asp on phagocytic activity of circulating granulocytes in rat models of Aβ25-35-induced AD (n=5 in all groups). FIG. 29A is the number of cells performing phagocytosis; FIG. 29B is phagocytic activity. Note: *p<0.05 vs. intact animals, ^{#}p<0.05 vs. control animal models of AD

FIG. 30A and FIG. 30B show therapeutic effects of ⁶⁴Zn-asp on phagocytic activity of circulating monocytes in rat models of Aβ25-35-induced AD (n=5 in all groups). FIG. 30A is a relative number of cells performing phagocytosis; FIG. 30B is phagocytic activity.

Note: *p<0.05 vs. intact animals, ^{#}p<0.05 vs. control animal models of AD

FIG. 31A and FIG. 31B show the effects of ⁶⁴Zn-asp on the oxidative metabolism in circulating granulocytes (FIG. 31A) and monocytes (FIG. 31B) in rat models of Aβ25-35-induced AD. Note: *p<0.05 vs. intact animals; ^{#}<0.05 vs. untreated AD animal models

FIG. 32A and FIG. 32B show the expression of CD86 in the population of circulating phagocytes in ⁶⁴Zn-asp treated rat models of Alzheimer's disease induced by the infusion of Aβ25-35 (n=5 in all groups). FIG. 32A is the number of expressing cells in the population analyzed; FIG. 32B is the level of expression. *p<0.05 versus intact animals; ^{#}≤0.05 versus control AD animal models

FIG. 33A and FIG. 33B show the expression of CD206 in the population of circulating phagocytes in ⁶⁴Zn-asp treated rat models of Alzheimer's disease induced by the infusion of Aβ25-35 (n=5 in all groups). FIG. 33A is the number of expressing cells in the population analyzed; FIG. 33B is the level of expression. *p<0.05 versus intact animals; ^{#}≤0.05 versus control AD animal models

### DETAILED DESCRIPTION

As used herein, the word “a” or “plurality” before a noun represents one or more of the particular noun.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

“Effective amount,” “prophylactically effective amount,” or “therapeutically effective amount” refers to an amount of an agent or composition that provides a beneficial effect or favorable result to a subject, or alternatively, an amount of an agent or composition that exhibits the desired in vivo or in vitro activity. “Effective amount,” “prophylactically effective amount,” or “therapeutically effective amount” refers to an amount of an agent or composition that provides the desired biological, therapeutic, and/or prophylactic result. That result can be reduction, amelioration, palliation, lessening, delaying, and/or alleviation of one or more of the signs, symptoms, or causes of a disease, disorder or condition in a patient/subject, or any other desired alteration of a biological system. An effective amount can be administered in one or more administrations.

An “effective amount,” “prophylactically effective amount,” or “therapeutically effective amount” may be first estimated either in accordance with cell culture assays or using animal models, typically mice, rats, guinea pigs, rabbits, dogs or pigs. An animal model may be used to determine an appropriate concentration range and route of administration. Such information can then be used to determine appropriate doses and routes of administration for humans. When calculating a human equivalent dose, a conversion table such as that provided in Guidance for Industry: Estimating the Maximum Safe Starting Dose in Initial Clinical Trials for Therapeutics in Adult Healthy Volunteers (U.S. Department of Health and Human Services, Food and Drug Administration, Center for Drug Evaluation and Research (CDER), July 2005) may be used. The person of ordinary skill in the art is aware of additional guidance that may also be used to develop human therapeutic dosages based on non-human data. An effective dose is generally 0.01 mg/kg to 2000 mg/kg of an active agent, preferably 0.05 mg/kg to 500 mg/kg of an active agent. An exact effective dose will depend on the severity of the disease, patient's general state of health, age, body weight and sex, nutrition, time and frequency of administration, combination(s) of medicines, response sensitivity and tolerance/response to administration and other factors that will be taken into account by a person skilled in the art when determining the dosage and route of administration for a particular patient based on his/her knowledge of the art.

Such dose may be determined by conducting routine experiments and at the physician's discretion. Effective doses will also vary depending on the possibility of their combined use with other therapeutic procedures, such as the use of other agents.

As used herein, a “patient” and a “subject” are interchangeable terms and may refer to a human patient/subject, a dog, a cat, a non-human primate, etc.

All ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should be considered to include the end points 5 and 10.

It is further to be understood that the feature or features of one embodiment may generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### Alzheimer's Disease (“AD”)

The amyloid or “senile” plaques are the main factors in the pathophysiology of AD. The amyloid or “senile” plaques predominantly consist of Aβ peptides derived from the proteolytic processing of the amyloid precursor protein (APP). APP is a glycosylated transmembrane protein with a large N-terminal extracellular domain, a single hydrophobic transmembrane domain, and a small C-terminal cytoplasmic domain.

APP can be processed by one of two pathways: the amyloidogenic pathway, leading to the production of Aβ, and the non-amyloidogenic pathway. Chow V W et al., Neuromolecular Medicine 2010; 12(1):1-12. The predominant APP-processing pathway in healthy brain is the nonamyloidogenic pathway, where APP is cleaved by the α-secretase within the Aβ region, forming the secreted APPα (sAPPα) fragment and the membrane-bound C-terminal fragment of 83 amino acids (C83). α-secretase activity is attributed to the disintegrin and metalloprotease (ADAM) family of zinc metalloproteases, as they have a long zinc-binding consensus sequence. Bode W, et al., Adv Exp Med Biol 1996; 389:1-11. doi:10.1007/978-1-4613-0335-0_1. C83 is subsequently cleaved by the γ-secretase complex, generating APP intracellular domain (AICD) and p3. In the amyloidogenic pathway, APP is sequentially cleaved by the aspartyl protease, forming the secreted APPβ (sAPPβ) fragment and a membrane bound C-terminal fragment of 99 amino acids (C99). The C99 fragment is then further processed by the γ-secretase complex into AICD and Aβ peptides, predominantly 40 and 42 amino acids in length. It is these aggregation-prone Aβ peptides which form oligomeric and fibrillar structures which deposit in the brain and over time cause AD. Zhang Y W et al., Mol Brain 2011; 4:3. Published 2011 Jan. 7. doi:10.1186/1756-6606-4-3.

In a healthy brain, the relatively small amount of Aβ being produced constitutively is rendered safe by Aβ degrading enzymes. A large number of candidate Aβ degrading enzymes have been identified, with the majority being zinc metalloproteases. Bateman R J et al., Nature Medicine 2006; 12(7):856-861.

Zinc is the most abundant trace metal in the brain and it has multifactorial functions in Alzheimer's disease (AD). Zinc is critical in the enzymatic nonamyloidogenic processing of the amyloid precursor protein (APP) and in the enzymatic degradation of the amyloid-β (Aβ) peptide. Zinc binds to Aβ, promoting its aggregation into neurotoxic species; disruption of zinc homeostasis in the brain results in synaptic and memory deficits. A specific binding site for zinc has been localized in the cysteine-rich region (within the extracellular domain) on the ectodomain of APP. Bush A I et al., The Journal of Biological Chemistry. 1993; 268(22):16109-16112; Bush A I, et al., The Journal of Biological Chemistry 1994; 269(43):26618-26621. Clinical observations have shown that the zinc values in serum of AD patients are significantly lower compared to healthy controls.

High free copper has shown promising evidence in favor of association with etiology of AD. Free copper generates reactive oxygen species, resulting in activation of neuroinflammation and neurodegeneration. Neuroinflammation plays a significant role in the pathophysiology of AD, as well as other neurodegenerative diseases of the groups of synucleinopathies and tauopathies. Zhang F, Jiang L. Neuropsychiatr Dis Treat 2015; 11:243-256. doi:10.2147/NDT.S75546. Zinc therapy is considered a promising approach to the treatment of free copper toxicosis. Zinc induces the production of intestinal metallothioneins, which leads to increased excretion of free copper via the stool. Avan A, Hoogenraad TU. Journal of Alzheimer's Disease 46 (2015) 89-92 DOI 10.3233/JAD-150186. Zinc may have a role in sustaining the adhesiveness of APP during cell-cell and cell-matrix interactions. Multhaup G et al., FEBS Letters 1994; 355(2):151-154; Multhaup G, et al., Biochemistry 1998; 37(20):7224-7230.

### Methods and Compositions

In one aspect, this disclosure provides a method of treating or delaying the onset (i.e., preventing) AD in a patient in need thereof, comprising administering to the patient a pharmaceutical composition comprising zinc, at a therapeutically effective or a prophylactically effective dose for treating or preventing the disease. In some embodiments, the composition comprises ⁶⁴Zn-enriched zinc (the term “⁶⁴Znₑ” is used herein to refer to ⁶⁴Zn-enriched zinc).

In some embodiments, the solution comprising natural ⁶⁴Znₑ salt. In some embodiments, the ⁶⁴Znₑ salt is a citrate or aspartate. In some embodiments, the ⁶⁴Znₑ salt is ⁶⁴Znₑ aspartate with 2 molecules of aspartic acid.

In some embodiments, the ⁶⁴Zn-enriched zinc is in the form of a ⁶⁴Znₑ compound or a ⁶⁴Znₑ salt. In certain embodiments, the disclosed compositions contain zinc that is at least 80% ⁶⁴Znₑ, at least 90% ⁶⁴Znₑ, at least 95% ⁶⁴Znₑ, or at least 99% ⁶⁴Znₑ, for example, zinc that is 80% ⁶⁴Znₑ, 85% ⁶⁴Znₑ, 90% ⁶⁴Znₑ, 95% ⁶⁴Znₑ, 99% ⁶⁴Znₑ, or 99.9% ⁶⁴Znₑ.

In some embodiments, the ⁶⁴Znₑ is in a form of salt selected from the group consisting of aspartate (chemical formula —C₄H₅O₄N⁶⁴Znₑ) with 2 aspartic acid molecules, sulfate, and citrate.

In some embodiments, the ⁶⁴Znₑ is in a form of ⁶⁴Znₑ aspartate (chemical formula —C₄H₅O₄N⁶⁴Znₑ) with 2 aspartic acid molecules.

The term “⁶⁴Znₑ” is used herein to refer to ⁶⁴Zn-enriched zinc. That is, zinc that is enriched for ⁶⁴Zn such that ⁶⁴Zn is enriched greater than its usual percentage in zinc in nature.

Zinc in the form of the light isotope ⁶⁴Znₑ is absorbed in the body much better than naturally-occurring zinc. In certain embodiments, the disclosed compositions contain zinc that is at least 80% ⁶⁴Znₑ, at least 90% ⁶⁴Znₑ, at least 95% ⁶⁴Znₑ, or at least 99% ⁶⁴Znₑ, for example, zinc that is 80% ⁶⁴Znₑ, 85% ⁶⁴Znₑ, 90% ⁶⁴Znₑ, 95% ⁶⁴Znₑ, 99% ⁶⁴Znₑ, or 99.9% ⁶⁴Znₑ.

In some embodiments, the composition contains between 0.05 mg and 110 mg of ⁶⁴Znₑ. In some embodiments, the composition contains between 1 and 10 mg of ⁶⁴Znₑ. In some embodiments, the ⁶⁴Znₑ compound or a salt thereof is at least 90% ⁶⁴Znₑ and the composition is an aqueous solution in which ⁶⁴Znₑ is present at a concentration of between 0.1 mg/ml and 10 mg/ml.

In some embodiments, therapeutic doses for a human subject are between 0.2 and 0.8 mg of Zn-64 per kg of body weight of the human subject.

In some embodiments, the composition or solution is administered by injection. In other embodiments, the composition or solution is administered orally.

### Formulating and Administering Compositions

The disclosed composition may be administered to a subject in need thereof by any suitable mode of administration, any suitable frequency, and at any suitable, effective dosage.

In some embodiments, the total amount of zinc administered is the same as the U.S. recommended daily allowance or intake of zinc. In some embodiments, the total amount of Zn administered is ½, twice, three times, five times, or ten times the U.S. recommended daily allowance or intake of zinc. In some embodiments, the total amount of Zn is between ½ and 10 times the U.S. recommended daily allowance or intake of zinc. A composition for use in a disclosed method may comprise the prescribed daily amount to be administered once a day or some fraction thereof to be administered a corresponding number of times per day. A composition for use in a disclosed method may also comprise an amount of Zn to be administered once every two days, once every three days, once a week, or at any other suitable frequency.

The composition for use in a disclosed method may be in any suitable form and may be formulated for any suitable means of delivery. In some embodiments, the composition for use in a disclosed method is provided in a form suitable for oral administration, such as a tablet, pill, lozenge, capsule, liquid suspension, liquid solution, or any other conventional oral dosage form. The oral dosage forms may provide immediate release, delayed release, sustained release, or enteric release, and, if appropriate, comprise one or more coating. In some embodiments, the disclosed composition is provided in a form suitable for injection, such as subcutaneous, intramuscular, intravenous, intraperitoneal, or any other route of injection. In some embodiments, compositions for injection are provided in sterile and/or non-pyrogenic form and may contain preservatives and/or other suitable excipients, such as sucrose, sodium phosphate dibasic heptahydrate or other suitable buffer, a pH-adjusting agent such as hydrochloric acid or sodium hydroxide, and polysorbate 80 or other suitable detergent.

When provided in solution form, in some embodiments, the composition for use in a disclosed method is provided in a glass or plastic bottle, vial or ampoule, any of which may be suitable for either single or multiple use. The bottle, vial or ampoule containing the disclosed composition may be provided in kit form together with one or more needles of suitable gauge and/or one or more syringes, all of which preferably are sterile. Thus, in certain embodiments, a kit is provided comprising a liquid solution as described above, which is packaged in a suitable glass or plastic bottle, vial or ampoule and may further comprising one or more needles and/or one or more syringes. The kit may further comprise instruction for use.

In certain embodiments, the dosage of Zn is proportional to various authoritative daily ingestion guidance (e.g., recommended dietary allowance (USRDA), adequate intake (AI), recommended dietary intake (RDI)) of the corresponding element.

In some embodiments, the Zn dosage is between about ½ and about 20 times the guidance amount, more preferably between about 1 and about 10 times the guidance amount, even more preferably between about 1 and about 3 times the guidance amount. Thus, in certain embodiments, a single dose of a composition for use in a disclosed method for daily administration would be formulated to comprise a quantity within these ranges, such as about ½, about 1, about 3, about 5, about 10, and about 20 times the guidance amount. These amounts generally are for oral intake or topical application. In some embodiments, the intravenous dosage is lower, such as from about 1/10 to about ½ the guidance amount. Doses at the low end of these ranges are appropriate for anyone with a heightened sensitivity to a specific element or class of elements (e.g., those with kidney problems). For zinc, the daily guidance amount ranges from 2 mg in infants to 8-11 mg (depending on sex) for ages 9 and up. Daily dosages discussed throughout this application may be subdivided into fractional dosages and the fractional dosages administered the appropriate number of times per day to provide the total daily dosage amount (e.g. ½ the daily dose administered twice daily, ⅓ the daily dose administered three times daily, etc.).

The composition for use in a disclosed method can be produced by methods employed in accordance with general practice in the pharmaceutical industry, such as, for example, the methods illustrated in Remington: The Science and Practice of Pharmacy (Pharmaceutical Press; 21st revised ed. (2011) (hereinafter “Remington”).

In some embodiments, the composition for use in a disclosed method comprise at least one pharmaceutically acceptable vehicle or excipient. These include, for example, diluents, carriers, excipients, fillers, disintegrants, solubilizing agents, dispersing agents, preservatives, wetting agents, preservatives, stabilizers, buffering agents (e.g. phosphate, citrate, acetate, tartrate), suspending agents, emulsifiers, and penetration enhancing agents such as DMSO, as appropriate. The composition can also comprise suitable auxiliary substances, for example, solubilizing agents, dispersing agents, suspending agents and emulsifiers.

In certain embodiments, the composition further comprises suitable diluents, glidants, lubricants, acidulants, stabilizers, fillers, binders, plasticizers or release aids and other pharmaceutically acceptable excipients.

A complete description of pharmaceutically acceptable excipients can be found, for example, in Remington's Pharmaceutical Sciences (Mack Pub., Co., N.J. 1991) or other standard pharmaceutical science texts, such as the Handbook of Pharmaceutical Excipients (Shesky et al. eds., 8th ed. 2017).

In some embodiments, the composition for use in a disclosed method can be administered intragastrically, orally, intravenously, intraperitoneally or intramuscularly, but other routes of administration are also possible.

Water may be used as a carrier and diluent in the composition. The use of other pharmaceutically acceptable solvents and diluents in addition to or instead of water is also acceptable. In certain embodiments, deuterium-depleted water is used as a diluent.

Large macromolecules that are slowly metabolized, such as proteins, polysaccharides, polylactic acids, polyglycolic acids, polymeric amino acids, copolymers of amino acids, can also be used as carrier compounds for the composition. Pharmaceutically acceptable carriers in therapeutic compositions may additionally contain liquids, such as water, saline, glycerol or ethanol. Moreover, the said compositions may further comprise excipients, such as wetting agents or emulsifiers, buffering substances, and the like. Such excipients include, among others, diluents and carriers conventional in the art, and/or substances that promote penetration of the active compound into the cell, for example, DMSO, as well as preservatives and stabilizers.

The composition for use in a disclosed method may be presented in various dosage forms depending on the object of application; in particular, it may be formulated as a solution for injections.

The composition for use in a disclosed method may be administered systemically. Suitable routes of administration include, for example, oral or parenteral administration, such as intravenous, intraperitoneal, intragastric as well as via drinking water. However, depending on a dosage form, the disclosed composition may be administered by other routes.

In certain embodiments, the composition for use in a disclosed method comprising Zn is administered intragastrically at a concentration of 2.25 mg/ml.

In some embodiments, the composition for use in a disclosed method is about 2 ml.

In some embodiments, the level of enrichment of ⁶⁴Znₑ is about 99% or more. In other further embodiments, the ⁶⁴Znₑ of the 2 ml composition comprises or consists of zinc aspartate (chemical formula —C₄H₅O₄N⁶⁴Znₑ) with 2 aspartic acid molecules. The dose of the composition for use in a disclosed method may vary depending on the subject being treated, severity of the disease, the patient's condition and other factors that will be taken into account by a person skilled in the art when determining the dosage and route of administration for a particular patient based on his/her knowledge in the art.

Light isotopes may be purchased. Zn-64 oxide with the necessary degree of enrichment may be purchased from, for example, Oak Ridge National laboratory, Oak Ridge, TN, USA.

In some embodiments, zinc aspartate has a chemical formula —C₄H₅O₄N⁶⁴Znₑ, with 2 aspartic acid molecules. In some embodiments, the structure of zinc aspartate is:

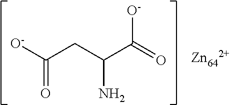

In certain embodiments, the composition for use in a disclosed method comprises ⁶⁴Znₑ at about 20% to about 100% of the composition.

The disclosed composition can be co-administered with another appropriate agent or therapy.

As shown in the Examples below, to study ⁶⁴Zn-aspartate (⁶⁴Zn-asp) for cognitive symptoms in rat models of AD, two models induced by infusion of a mixture of different beta-amyloid peptides—1-40 and 25-35—were used. ⁶⁴Zn-asp is referred in the Examples as “the test substance.”

Animal models of AD are a cornerstone for the drug development process and should be as relevant as possible for the disease, replicating its phenotype with a high degree of certainty. Over the past two decades, transgenic AD models contributed tremendously to the understanding of the molecular mechanisms involved in the onset and progression of the disease. However, numerous literature data show that the use of genetic models of AD does not allow reproducing a complete clinical picture of the disease, which is more reproducible when using rat models in which AD phenotype is induced by the infusion of amyloid beta peptide. Lecanu L, Papadopoulos V. Alzheimers Res Ther. 2013; 5(3):17. doi:10.1186/alzrt171. In such models, the AD phenotype is most often induced by administering a solution containing the human form of the 42 residue amyloid peptide (Aβ1-42) via the intracerebroventricular route. Mudò G et al., J Neuroinflammation 2019; 16(1):44. doi:10.1186/s12974-019-1417-4. Aβ₁₋₄₂ was chosen because of its excellent aggregating properties and because it was thought to constitute the nucleus of any amyloid plaque formation.

The AD model based on the infusion of beta-amyloid peptide 25-35 is a newer model. Among the Aβ fragments studied to date, the Aβ (25-35) peptide is the shortest Aβ fragment formed in vivo as a result of the action of brain proteases. Kubo, T. et al., J. Neurosci. Res. 2002; 70, 474-483. This peptide exhibits significant levels of molecular aggregation, retaining the toxicity of the full-sized peptide, although it is lacking in metal binding sites. In line with this finding, it has been proposed that the Aβ (25-35) peptide represents a biologically active region of Aβ.

Despite the fact that Aβ deposition in the central nervous system is a hallmark of AD and a possible cause of neurodegeneration, several reports have suggested that some non-aggregated amyloid molecules and their peptide fragments may intercalate into the plasma membrane of neurons and directly alter membrane activities. Pike, C. J. et al., J. Neurochem. 1995; 64, 253-265; Dahlgren, K. N. et al., J. Biol. Chem. 2002; 277, 32046-32053. Recent studies have demonstrated that in the earlier stages of AD, the non-aggregated form of Aβ fragments, namely, the mono/oligomeric Aβ (25-35) forms, is also able to penetrate through plasma membranes, causing intracellular toxicity mechanisms. Clementi M E et al., FEBS Lett. 2005; 579(13):2913-2918 doi:10.1016/j.febslet.2005.04.041.

In addition, the lag phase between β-amyloid (AD) deposition and neurodegeneration in Alzheimer's disease (AD) suggests that age-dependent factors are involved in the pathogenesis. Racemization of Ser and Asp in Aβ is a typical age-dependent modification in AD. It has been shown recently that Aβ1-40 racemized at Ser²⁶ ([D-Ser²⁶]Aβ1-40) is soluble and non-toxic to neuronal cells but is easily converted by brain proteases to truncated toxic fragments—[D-Ser²⁶]Aβ25-35/40. The immunohistochemical analyses using anti-[D-Ser²⁶]Aβ25-35/40-specific antibodies demonstrated the presence of [D-Ser²⁶]Aβ25-35/40 antigens in senile plaques and in degenerating hippocampal CA1 neurons in AD brains, but not in age-matched control brains. These results confirm the hypothesis that soluble [D-Ser²⁶]Aβ1-40, possibly produced during aging, is released from plaques and converted by proteolysis to toxic [D-Ser²⁶]Aβ25-35/40, which damage hippocampal CA1 neurons by enhancing excitotoxicity in AD. Kubo T. et al., J Neurosci Res. 2002; 70(3):474-483 doi:10.1002/jnr.10391.

Moreover, research has shown that a significant number of proteins and peptides can assemble into amyloid structures under experimental conditions. Although these polypeptides show neither conformational nor structural homology, their amyloid fibrils apparently have a common structural feature—the presence of a β-folded structure in the center, indicating that amyloid formation is a common property of the polypeptide backbone. Such a process may progress in the body if cellular mechanisms are not able to eliminate protein aggregates.

Another common feature of amyloid aggregates is that they develop by a nucleation-dependent mechanism and that the initial oligomeric and prefibrillar structures of various proteins are cytotoxic.

Mature fibrils are considered as an inert material, which can cause physical damage to organs and tissues. Moulias R et al., Ann Méd Interne (Paris) 2002; 153:441-445; Bucciantini M et al., Nature 2002; 416: 507-511.

An autoimmune component is recently described of the pathophysiology of dementia, including AD. Clinical studies have convincingly demonstrated that autoantibodies to various molecules are associated with the development and progression of AD. Thus, antibodies to Aβ and Tau protein, multiple transmitter and receptor molecules (glutamate, dopamine, etc.), glial markers such as GFAP, lipids (ceramides, oxidized low-density lipoproteins), vascular markers, such as RAGE (Receptor for Advanced Glycation End Products, a receptor involved in the pathogenesis of almost all neurodegenerative diseases), cellular enzymes such as aldolase and many other autoantigens are found in the serum of AD patients. Wu J, Li L. J Biomed Res. 2016; 30(5):361-372. doi:10.7555/JBR.30.20150131; MacLean M. et al., Neurochem Int. 2019; 126:154-164. doi:10.1016/j.neuint.2019.03.012. Although the role of these autoantibodies in AD remains unclear, their association with the development and progression of the disease convincingly proves the leading role of the immune system in its pathophysiology. According to the results of published clinical observations, Aβ 25-35 is one of the autoantigens to which high titers of antibodies are found in the serum of 90% of patients with AD. Gruden M A et al., Dement Geriatr Cogn Disord. 2004; 18(2):165-171 doi:10.1159/000079197. Thus, it made sense to use an AD model induced by the infusion of Aβ 25-35 herein.

### EXAMPLES

For this invention to be better understood, the following examples are set forth. These examples are for purposes of illustration only and are not to be construed as limiting the scope of the invention in any manner.

### Example 1 Therapeutic Effects of ⁶⁴Zn-Asp in Rat Model of AD

### Materials and Methods

### Experimental Animals

Male Wistar rats (300-500 g) were used. The animals were maintained under standard conditions in the vivarium of the ESC “Institute of Biology and Medicine” of Taras Shevchenko National University of Kyiv. They were given ad libitum access to food and water.

### Modelling Alzheimer's Disease in Rats

Alzheimer's Disease was induced in old male rats (14 months old) by giving them intrahippocampal injections of aggregated Amyloid Beta (Aβ 1-40) Peptide (human) (Cayman Chemical Company) and Amyloid Beta (Aβ 25-35) Peptide (human) (Tocris Bioscience, Brostol). Aβ1-40 and Aβ 25-35 were dissolved in bidistilled water to the concentration of 15 μmol/L and incubated at 37° C. for 24 hours for aggregation. Aβ conglomerates were broken up by ultrasound and sterilized immediately prior to injection.

Rats were anesthetized using a mixture of ketamine (75 mg/kg, diluted in sterile water for injection, Sigma, USA) and 2% xylazine (100 μl/rat, Alfasan International B.V., Netherlands) administered intraperitoneally in a total volume of 1 ml. A rat was placed in a stereotaxic apparatus (SEZH-4) modified for rats. The animal was then scalped from the point of intersection of the sagittal suture with bregma (zero point): 2 mm distally, 2 mm laterally and 3.5 mm in depth and a trephine opening was made with an injection needle, directly into the hippocampus. Dissolved Aβ1-40 or Aβ25-35 was collected into a home-made microinjector and its tip was dropped into the trephine opening.

The suspension in a volume of 10 μl per animal was infused for 5 minutes at a rate of 0.5 μl/minute (every 15 seconds). After Aβ was administered, the tip of the microinjector remained in the brain tissue for 4 minutes. The microinjector was then removed and stitches were put on the scalp soft tissues of the animal. Control animals were administered placebo, 10 μl sterile deuterium-depleted water, instead of Aβ1-40 or Aβ25-35 (sham operated animals).

### Experimental Design

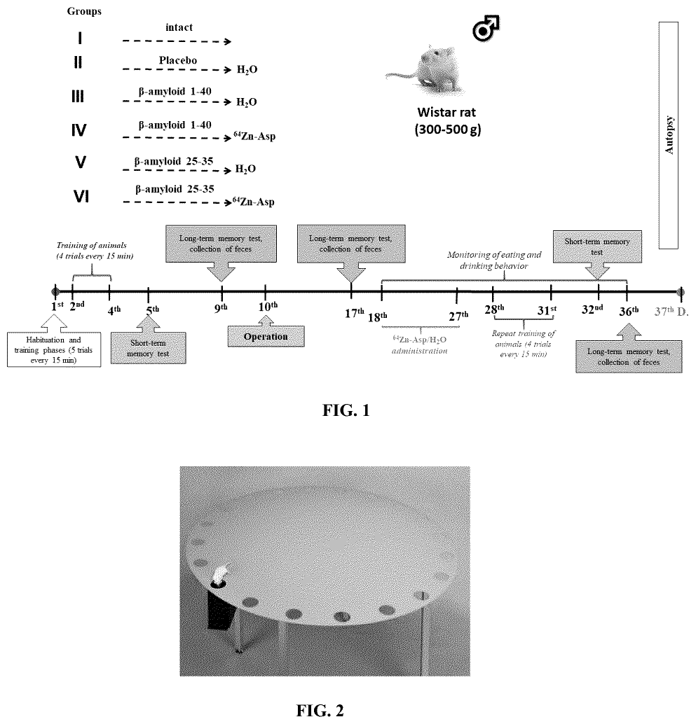

Animals were distributed into 6 groups:
I (n=7)—intact animals that were maintained under standard vivarium conditions and did not receive any manipulations;
II (n=7)—sham operated rats that were administered daily 0.1 ml of deuterium-depleted water intravenously (i.v.) for 10 days after the operation (from the 18^{th} to 27^{th} day of the experiment inclusively);
III (n=7)—rats that were daily administered 0.1 ml of deuterium-depleted water (i.v.) for 10 days (from the 18^{th} to 27^{th} day of the experiment inclusively) after they were infused Aβ1-40 to induce AD;
IV (n=7)—rats in which AD was induced by the infusion of Aβ1-40 and which were then administered daily a solution of ⁶⁴Zn-asp in a volume of 1.5 mg/kg, i.v., for 10 days (from the 18^{th} to 27^{th} day of the experiment inclusively);
V (n=7)—rats in which AD was induced by the of Aβ25-35 and which were then daily administered 0.1 ml of deuterium-depleted water (i.v.) for 10 days (from the 18^{th} to 27^{th} day of the experiment inclusively);
VI (n=7)—rats in which AD was induced by the infusion of Aβ25-35 and which were then daily administered a solution of ⁶⁴Zn-asp in a volume of 1.5 mg/kg, i.v., for 10 days (from the 18^{th} to 27^{th} day of the experiment inclusively) (FIG. 1).

### Assessment of Eating and Drinking Behaviors in Rats

The animals were placed in individual cages and the amounts of food and water consumed were measured daily for each rat, starting from the 18^{th} day (8 days after surgery) and until the end of the experiment (37^{th} day). The data were first averaged into 1 rat per day within a group, and then into 1 rat per day for the entire period of observation.

### Immunohistochemical Identification of Dopaminergic Neurons

Degeneration of the hippocampal neurons was assessed using immunohistochemical analysis for the expression level of tyrosine hydroxylase (TH). Immunohistochemical staining was performed using primary anti-TH antibodies at a 1:200 dilution (Millipore, AB152). Endogenous peroxidase activity was blocked with a blocking reagent (Dako, EnVision Flex, DM821). Nonspecific antibody binding was blocked using 4% dry milk dissolved in Tris-buffered saline (TBS) containing 0.2% Triton X-100.

The primary antibody was diluted in TBS containing 0.2% Triton X-100 and applied to tissue sections. The sections were then incubated overnight (+4° C.). Secondary antibodies (anti-rabbit biotinylated antibodies, 1:200) were incubated for 60 minutes. The immunoreaction was developed using diaminobenzidine (Dako, EnVision) applied for 5 minutes. The results of immunohistochemical staining were evaluated at the light-optical level using a Zeiss Primo Star microscope. The intensity of TH-positive staining was evaluated using a semiquantitative scoring system as described in the Quantitative Scoring Method ([http://www.ihcworld.com/ihc_scoring.htm]) taking into account the number of positive (stained) cells and the intensity of staining (Table 1). The results were presented as a quick score (Q) calculated by the following formula: Q=P×I, where P is the percentage of positive cells and I is the staining intensity.

TABLE 1
Semi-quantitative scale of scores to assess
TH-positive staining intensity

| Scores | 0 | 1 | 2 | 3 | 4 |
|---|---|---|---|---|---|
| Percentage of | <10% | 10-25% | 25-50% | 50-75% | >75% |
| positive cells | | | | | |
| Intensity of | No stain | Weak | Moderate | Strong | — |
| staining | | staining | staining | staining | |

### Short-Term and Long-Term Memory Change Tests Using Barnes Maze

The Barnes maze (FIG. 2) is a tool used to measure spatial learning and memory of a rodent and helps to identify cognitive deficits in rodents that model for disease such as Alzheimer's disease. Kinga Gawel et al., Assessment of spatial learning and memory in the Barnes maze task in rodents-methodological consideration, Naunyn Schmiedebergs Arch Pharmacol. 2019; 392(1): 1-18. doi: 10.1007/s00210-018-1589-y. The test was first developed by Dr. Carol Barnes in 1979 and is based on the intrinsic inclination of the subjects to escape from an aversive environment, which is a brightly lit open surface, and seek shelter in a small dark “escape box”. An animal learns to remember the location of the target zone (“escape box”) using peripheral visual cues in the testing area as reference points. In this test, the animal finds out the location of the “escape box” under stressless conditions, in contrast to other tests that utilize a strong aversive stimulus (stress induced by swimming) or deprivation (food or water deprivation) as reinforcement to increase behavioral variability based on individual characteristics of the animal.

Barnes maze used consisted of a circular table with 16 circular holes around its circumference. Peripheral visual cues, such as black marks (a triangle on one wall and two parallel strips on the opposite wall), were placed for better orientation of the animal. Under one of the holes an “escape box” containing standard filler for animals was fixed. Each animal was assigned its number of the hole to which the “escape box” was fixed. The other holes remained open. Before performing the probe phase (after the operation), the number of the hole was changed.

On the 1^{st} day of the experiment, during the first training of rats, a habituation session was performed, which was not repeated during the probe phase. A rat was placed in the center of the circular table and kept under a non-transparent hood for 10 seconds. The light above the table was then turned on and the hood lifted. The rat was allowed to move freely around the table for 2-3 minutes. If the animal did not manage to find the “escape box” during this time, it was helped to find the right path.

Fifteen minutes after the habituation session, on the 2^{nd}, 3^{rd} and 4^{th} days of the experiment, the task training was repeated 4 times every 15 minutes (min) (3 min on the table surface+1 min in the “escape box” with the light off). The time during which the animal managed to reach the “escape box” was recorded at each trial.

Short-term memory of the animals was tested on the 5^{th} day, and long-term memory was tested on the 9^{th} day (5^{th} day after the last training). All the holes of the maze were closed, and the rat was allowed to freely explore the open arena for 90 seconds to find “its” corresponding hole on the table top (under which the “escape box” was previously located) based on previously acquired skills. The amount of time the animal spent searching for the correct hole, and the time it spent near the hole was recorded. The table surface was disinfected after each trial.

18 days after the operation, the animals had another 4-day training session (probe phase) with a changed location of the “escape box”. As in the case before the operation, short-term memory was tested on the 5^{th} day, and long-term memory was tested on the 9^{th} day (5^{th} day after the last training session).

The following time periods were measured in seconds: 1) the time the animal spent to find the entrance to the “escape box” (assessment of spatial learning and spatial memory associated with the hippocampal function); 2) the time the animal spent near the entrance (assessment of cognitive flexibility associated with the function of the frontal cortex of the brain)—the less time the animal spends near the closed entrance, the quicker it understands that it is necessary to look for escape elsewhere.

### Assessment of Soluble Amyloid Beta and Pau Protein Levels in Hippocampal Homogenates

The levels of soluble forms of amyloid beta and tau protein in the hippocampal homogenates of rat models of AD were measured using ELISA kits in accordance with the manufacturer's recommendations. Hippocampal homogenates were also prepared in accordance with the manufacturer's recommendations. A complex of protease and phosphatase inhibitors was used to prevent proteolytic degradation of amyloid beta in the homogenate.

### Hematologic Study

The blood count values were analyzed at the completion of the experiment (day 37). The absolute number of leukocytes, as well as the absolute and relative numbers of lymphocytes, monocytes and neutrophilic granulocytes were calculated.

### Assessment of Endocytic Activity of Phagocytes of Various Localization

The phagocytic activity of microglia and peripheral blood phagocytes was analyzed on a flow cytometer using FITC-labeled S. aureus Wood 46 cells as an object of phagocytosis. The S. aureus cells were obtained from the collection of microorganisms of the Department of Microbiology and Immunology of the ERC Institute of Biology and Medicine of the National Taras Shevchenko University. Differential assessment of phagocytic activity values of circulating mono- and polymorphonuclear phagocytes was carried out using a gating method.

### Assessment of Oxidative Metabolism of Phagocytes of Various Localization

Oxidative metabolism of phagocytes of various localization was analyzed by flow cytometry using the cell-permeant 2′7′-dichlorodihydrofluorescein-diacetate (DHP) (carboxy-H₂DCFDA, Invitrogen, USA) which is converted by intracellular esterases to the nonfluorescent membrane-impermeable carboxy-H₂DCF form. Differential assessment of oxidative metabolism values of circulating mono- and polymorphonuclear phagocytes was carried out using a gating method. To assess a metabolic reserve the cells were treated with LPS (Sigma, USA).

### Assessment of Phenotypic Profile of Phagocytes of Various Localization

The phenotypic profile of phagocytes of various localization was characterized by the expression of markers of functional maturity and metabolic polarization (CD206 and CD86), which was determined by flow cytometry and the use of monoclonal antibodies of appropriate specificity marked with fluorescent dyes (Abcam, Becton Dickinson).

### Statistical Data Analysis Methods

The numerical results were processed using statistical data analysis methods using Statistica 12.0 software package. To determine statistical significance of the reliable difference between the results shown by each group, the Student's t-test was used. Significance was set at p<0.05.

### Results of the Study

### Therapeutic Effects of ⁶⁴Zn-Asp on Cognitive Activity as Well as Local and Systemic Immune Reactivity in Rat Models of Alzheimer's Disease Induced by Infusion of Aβ1-40

### Effects of ⁶⁴Zn-Asp on Cognitive Symptoms in Rat Models of Aβ1-40-Induced Alzheimer's Disease

### Effects of ⁶⁴Zn-Asp on Body Weight Changes in Rat Models of Aβ1-40-Induced Alzheimer's Disease

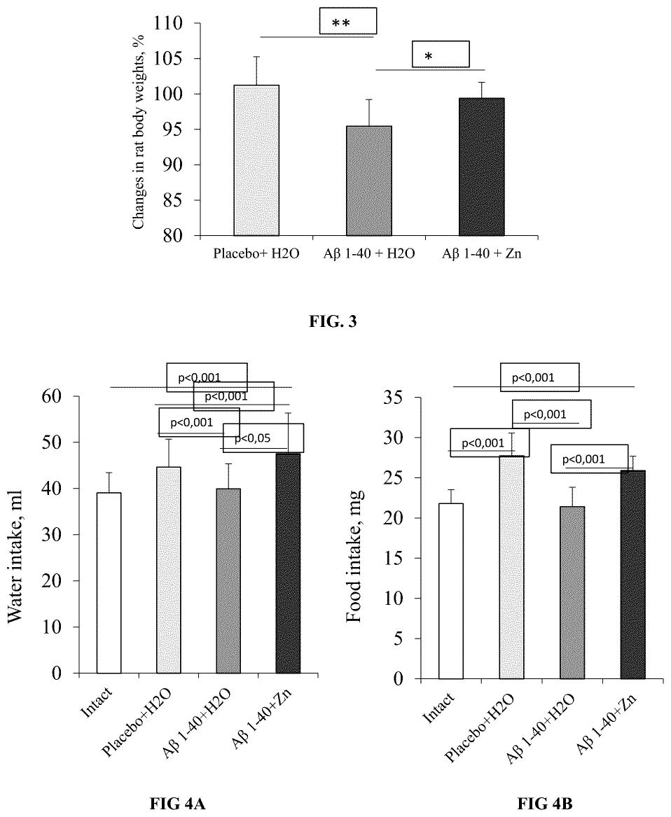

Animal body weight is a classic clinical sign that characterizes the general condition of an animal, and its loss during the experiment indicates that the condition of the animal is deteriorating. Since this experiment involved old animals with an initial body weight of 350-500 g, changes in their body weights during the experiment were insignificant in comparison with young animals (120-200 g) for the same period of time. Despite this fact, a significant loss in the body weight of animal models of Aβ1-40-induced AD within 1 month of the experiment was observed. The initial animal body weight in the group of sham-operated animals (prior to the operation) was 445.0±41.1 g, and at the end of the experiment, on the day of autopsy, it was 449.5±37.4 g, i.e. the weight gain was 1.3±4.0%. The body weight of rat models of Aβ1-40-induced AD before the operation was 361.1±25.3 g, and on the day of autopsy it was 340.3±33.5 g, which indicates a weight loss of 4.3±3.7% (P<0.01 compared with sham-operated animals) (FIG. 3).

Administration of ⁶⁴Zn-asp significantly improved this parameter. Thus, the body weight of ⁶⁴Zn-asp-treated rats of rat models of Aβ1-40-induced Alzheimer's disease before the operation was 432.1±29.7 g, and at the end of the experiment, on the day of autopsy, it was 425.4±40.8 g, which reflects the body weight loss in this experimental group by 0.6±2.3% (P<0.05, compared with sham-operated animals).

### Effects of ⁶⁴Zn-Asp on Eating and Drinking Behavior in Rat Models of Aβ1-40-Induced Alzheimer's Disease

Changes in body weight of rat models of Aβ1-40-induced AD were associated with a decrease in their food and water intake compared with the sham-operated animals (FIG. 4A and FIG. 4B).

⁶⁴Zn-asp-treated rat models of Aβ1-40-induced Alzheimer's disease were observed to restore their eating and drinking behavior to normal.

### Effects of ⁶⁴Zn-Asp on Tyrosine Hydroxylase Expression in the Hippocampus of Rat Models of Aβ1-40-Induced Alzheimer's Disease

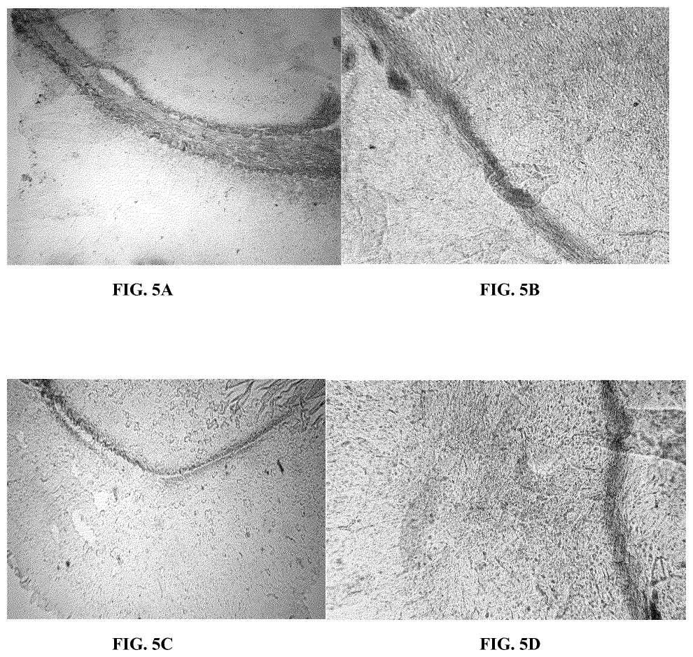

According to the result of immunohistochemical analysis of hippocampal slice preparations from intact animals their level of expression of tyrosine hydroxylase was 6.0±0.0 scores. In sham-operated animals, the staining intensity of TH-positive cells did not differ significantly from that of intact rats and was 7.0±1.7 scores (Table 2). The expression of tyrosine hydroxylase in rat models of Aβ1-40-induced AD was 2.3±1.5 scores, which is significantly lower than the values obtained from intact and sham-operated animals and is indicative of the destruction of hippocampal dopaminergic neurons during AD. Administration of ⁶⁴Zn-asp to rat models of Aβ1-40-induced AD caused an increase in their Q to 4.0±2.0 scores, mainly due to an increase in the intensity of immunopositively stained cells rather than in their number compared to untreated AD rat modes and sham-operated rats, and almost returned this parameter to control values (FIG. 5A-FIG. 5D).

TABLE 2
Staining intensity and percentage of TH-positive hippocampal cells in
rat models of Aβ1-40-induced Alzheimer's disease

| | Animal | Assessment | Assessment (intensity | |
|---|---|---|---|---|
| | number | (positive cells, P) | of staining, I) | Q = P × I |
| Intact | 658 E | 3 | 2 | 6 |
| animals | 658 F | 3 | 2 | 6 |
| | 658G | 3 | 2 | 6 |
| Placebo | 659 A | 3 | 2 | 6 |
| (H₂O) | 659 B | 3 | 3 | 9 |
| | 659 C | 3 | 2 | 6 |
| Aβ 1-40 + | 660 E | 2 | 2 | 4 |
| H₂O | 660 F | 2 | 1 | 2 |
| | 660 G | 1 | 1 | 1 |
| Aβ 1-40 + | 661 E | 2 | 2 | 4 |
| Zn | 661 F | 1 | 2 | 2 |
| | 661 G | 2 | 3 | 6 |

The results obtained indicate a protective role of the test substance in relation to the functions of dopaminergic neurons in the hippocampus.

### Effects of ⁶⁴Zn-Asp on the Spatial Memory in Rat Models of Aβ1-40-Induced Alzheimer's Disease

Alzheimer's disease, which is most common in people of old age, is associated with impairment of declarative memory, memory of events. Declarative memory in humans has parallels with the spatial memory in rodents (and this is why rodents provide a good model of declarative memory). Neurons responsible for declarative memory have representation in the hippocampus and are associated with a specific neuronal process known as long-term potentiation. In rodents, the hippocampus is involved in coding of spatial information which is studied in various mazes. The Barnes maze is used.

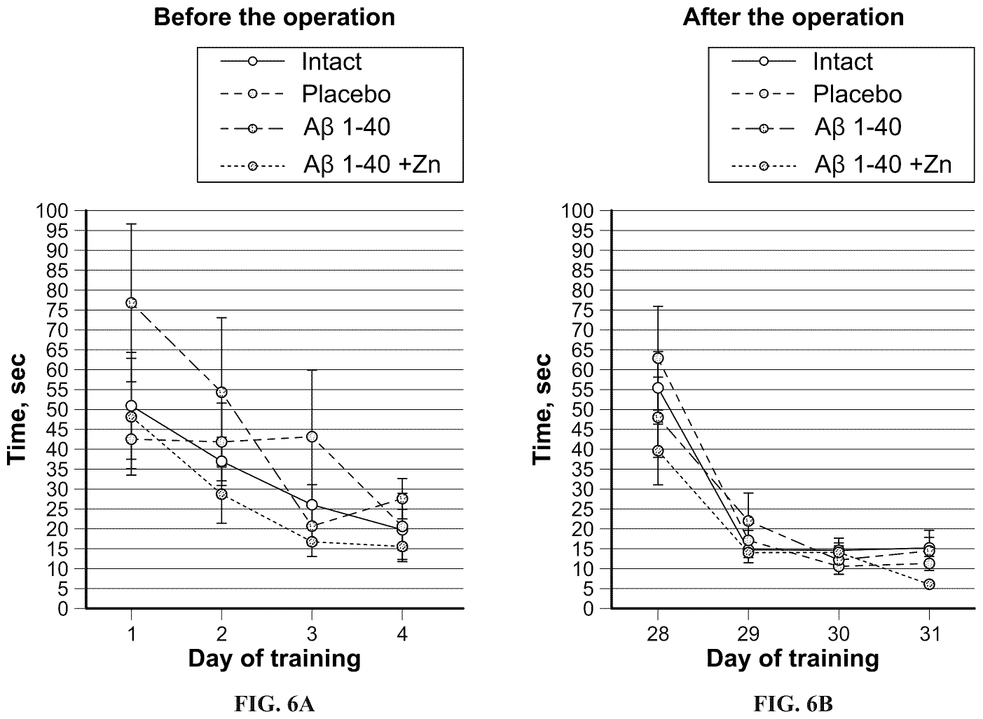

To assess the time spent on spatial learning in Alzheimer's disease, the time spent to find the “escape box” during 4 days of training before the operation and during 4 days of training after the operation (starting from day 18 after the operation) was compared. Different locations of the “escape box” was used in the training and probe phases before and after surgery. As can be seen in FIG. 6, during 4-day trainings, both before and after surgery, all groups of rats reduced the time spent to find an entrance to the escape hole. No difference in time was found between the control groups (intact and placebo-treated animals) and rat models of Aβ1-40-induced Alzheimer's disease.

To assess short-term memory, 24 hours after the last 4-day training phase (on the 5^{th} day after the start of training), the rats were placed in the Barnes maze, but the entrance to the “escape box” was closed. To assess long-term memory, the same test was repeated on the 5^{th} day following the 4-day training phase.

Time spent by each animal to find the “escape box” was measured. The faster the animal found the correct hole, the higher was the level of its spatial memory (the hippocampal function). The level of cognitive flexibility was also assessed by measuring time the animal spent near the entrance to the escape hole—the less time the animal was there, the higher level of cognitive flexibility it possessed (the frontal cortex function), i.e., the animal realized faster that the escape hole should be sought elsewhere.

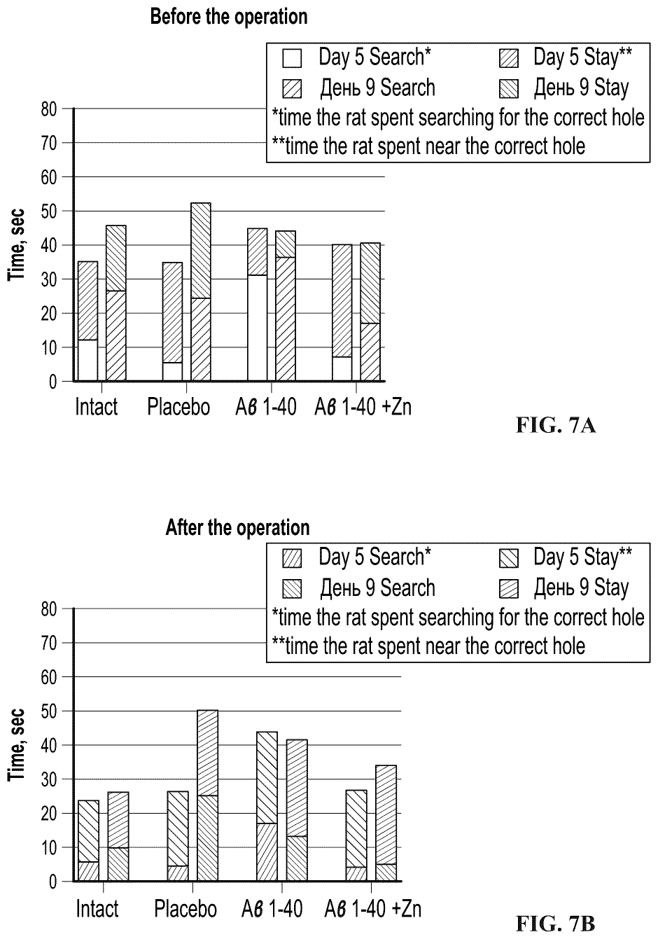

As is seen from Tables 3. and FIG. 7A and FIG. 7B, the values shown by animals from all groups before the operation were rather individual, therefore it was logical to compare patterns of changes rather than absolute numbers.

As shown in Table 3 and FIG. FIG. 7A and FIG. 7B, the time rats from all groups spent searching for the “escape box” before the operation naturally increased between the tests for this parameter 24 hours and 5 days after the training trials.

TABLE 3
Effects of ⁶⁴Zn-asp on the spatial short-term and long-term memory
efficiency (time spent to find an entrance to the “escape box”) in rat models of
Aβ1-40-induced Alzheimer's disease, M ± SD

| | Short-term memory | | | Long-term memory | | |
|---|---|---|---|---|---|---|
| | 24 hours following training | | | 5 days following training | | |
| | Time spent to find the | | | Time spent to find the | | |
| | “escape box”, sec | | | “escape box”, sec | | |
| | before | after | % of | before | after | % of |
| Groups | surgery | surgery | changes | surgery | surgery | changes |
| Intact rats, | 12.14 ± 04.03 | 5.71 ± 1.25 | −52.94 | 26.57 ± 06.5 | 9.86 ± 04.29 | −62.9 |
| n = 7 | | | (p = 0.37) | | | (p = 0.13) |
| Sham- | 5.5 ± 1.31 | 4.5 ± 1.06 | −18.18 | 24.33 ± 9.17 | 25.17 ± 13.35 | 0 |
| operated rats, | | | (p = 0.27) | | | |
| n = 7 | | | | | | |
| Aβ1-40 + H₂O, | 31.17 ± 7.28 | 17 ± 9.19 | −45.46 | 36.33 ± 17.26 | 13.17 ± 3.25 | −63.76 |
| n = 7 | | | (p = 0.13) | | | (p = 0.68) |
| Aβ1-40 + ⁶⁴Zn- | 7.14 ± 1.49 | 4.14 ± 0.74 | −42.04 | 17 ± 6.74 | 5 ± 1.54 | −70.59 |
| asp, n = 7 | | | (p = 0.37) | | | (p = 68) |

When assessing the level of cognitive flexibility in rats by the time the animal spent near the escape hole, a natural decrease was observed in this parameter 24 hours and 5 days after the 4-day training phase, and this pattern was typical for all groups of animals before the operation (Table 4, FIG. 7A and FIG. 7B).

During the probe phase (18 days after surgery), when the animals' short-term and long-term memories were tested, rats of all groups reduced time spent searching for the “escape box” by an average of 40% and 33%, respectively, in comparison with values within the group. It should be noted that rat models of Aβ1-40-induced AD showed the same pattern of changes in these parameters as intact animals, and similar changes were observed in the group of rat models of Aβ1-40-induced AD treated with ⁶⁴Zn-asp. It can therefore be concluded that there is no statistically significant impairment either in short-term or long-term memories in rat models of Aβ1-40-induced Alzheimer's disease.

In the probe phase, intact animals and sham-operated animals decreased time spent near the entrance to the “escape box” by an average of 23% 24 hours after the last training trial (short-term effect) and by an average of 12% 5 days after the last training trial (long-term effect). This indicates a normal level of cognitive flexibility in rats (Table 4). It should be noted that an opposite picture was observed in rat models of Aβ1-40-induced Alzheimer's disease: there was a 2-fold (P=0.02) increase in the time spent near the entrance to the “escape box” (short-term memory) and a stronger manifestation of this parameter when testing the long-term memory—a 4-fold increase in the time spent near the escape hole (P=0.04). This fact indicates impaired function of the frontal cortex responsible for cognitive functions in Aβ1-40-induced Alzheimer's disease.

TABLE 4
Effects of ⁶⁴Zn-asp on the cognitive flexibility in rat models of Aβ1-40-induced
Alzheimer's disease 24 hours and 5 days after the last training trial, M ± SD

| | Short-term memory | | | Long-term memory | | |
|---|---|---|---|---|---|---|
| | 24 hours following training | | | 5 days following training | | |
| | Time spent near the | | | Time spent near the | | |
| | entrance to the “escape | | | entrance to the “escape | | |
| | box”, sec | | | box”, sec | | |
| | before | after | % of | before | after | % of |
| Groups | surgery | surgery | changes | surgery | surgery | changes |
| Intact rats, | 23 ± 2.53 | 18 ± 2.18 | −21.74 | 19.14 ± 1.94 | 16.29 ± 2.31 | −14.93 |
| n = 7 | | | (p = 0.04) | | | (p = 0.31) |
| Sham- | 29.33 ± 2.55 | 21.83 ± 6.67 | −25.57 | 28 ± 5.3 | 25 ± 6.31 | −10.71 |
| operated rats, | | | (p = 0.22) | | | (p = 0.78) |
| n = 7 | | | | | | |
| Aβ1-40 + H₂O, | 13.67 ± 3.89 | 26.83 ± 12.19 | 96.27 | 7.71 ± 5.83 | 28.33 ± 7.82 | 67.28 |
| n = 7 | | | (p = 0.02) | | | (p = 0.04) |
| Aβ1-40 + ⁶⁴Zn- | 33 ± 7.39 | 22.57 ± 7.77 | −31.6 | 23.57 ± 6.61 | 29 ± 6.14 | 23.03 |
| asp, n = 7 | | | (p = 0.01) | | | (p = 0.58) |

Administration of ⁶⁴Zn-asp significantly improved the cognitive function in rat models of AD and virtually returned it to the values obtained from intact and sham-operated animals. Voikar V. Evaluation of methods and applications for behavioral profiling of transgenic mice. Academic dissertation. Faculty of Biosciences, University of Helsinki. 2006. 73 p.

Therapeutic Effects of ⁶⁴Zn-Asp on the Aβ and Tau Protein Levels in Hippocampal Homogenate from Rat Models of Alzheimer's Disease Induced by the Infusion of Aβ1-40

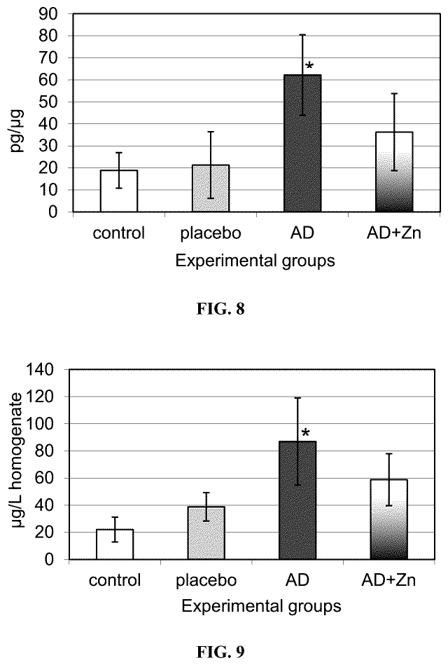

Presence of soluble Aβ and tau protein in the hippocampus is an unmistakable sign of AD development in animal models of AD and a marker for assessing the severity of disease and the effectiveness of pathogenetic methods of treatment. According to the results, the level of soluble Aβ in the hippocampal homogenates in rat models of AD induced by Aβ1-40 infusion was almost 4 times higher than that in intact animals (FIG. 8). In the placebo-treated animals (sham-operated animals), the level of soluble Aβ was also slightly higher than in intact rats. The level of Aβ in the hippocampus of AD rat models treated with the test substance was 1.5 times lower than in control AD rat models but did not reach the level in intact/sham-operated animals. It should be noted that this parameter showed exceptional variability, which made it impossible to adequately assess the evidence of the obtained data. Such high degree of variability could be due to a small statistical sampling of animals, a method of sample preparations for the test systems used in the study that differed from a similar procedure described in the literature for testing this parameter using the ELISA kits of other manufacturers (Xuan A et al., J Neuroinflammation 2012; 9:202. doi:10.1186/1742-2094-9-202; Wang L et al., Iran J Basic Med Sci. 2017; 20(5):474-480. doi:10.22038/IJBMS.2017.8669), as well as the natural variability of this parameter in AD models. It is possible that the concentration of insoluble forms of A3, recently described in the literature (Zhao H F et al., Neuroscience. 2015; 310:641-649 doi:10.1016/j.neuroscience.2015.10.006) may be a more adequate criterion for the formation of senile plaques in AD.

The level of phosphorylated tau protein in hippocampal homogenates from rat models of AD also exceeded the value obtained from intact animals more than 4-fold, which is a criterion for the disease progression and validity of the model (FIG. 9). In animals that received a therapeutic course of treatment with ⁶⁴Zn-asp, the levels of this protein in the hippocampus were lower than in control AD models, although the difference cannot be considered significant due to wide individual variability of this parameter in all groups of animals, most likely due to the above reasons.

In general, the analysis of the levels of proteins involved in the pathogenesis of AD shows that the test substance has a pathogenetic therapeutic effect resulting in a decrease in the concentration of plaque-forming components in AD animal models.

### Therapeutic Effects of ⁶⁴Zn-Asp on Functional and Phenotypic Properties of Microglia in Rat Models of Alzheimer's Disease Induced by the Infusion of Aβ1-40

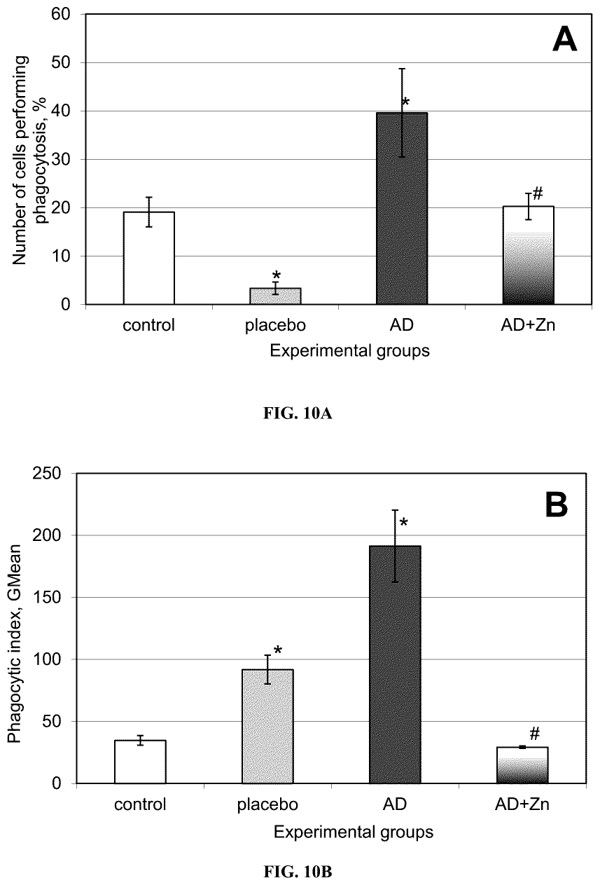

Phagocytic activity of microglia is an indicant of their activated state, any changes in which should be viewed in the context of changes in other functional and phenotypic characteristics. Increased phagocytic activity of microglial cells may accompany both pro- and anti-inflammatory microglial activation. In addition, with increased permeability of the blood brain barrier (BBB), the population of resident microglial cells is replenished by peripheral blood phagocytes, the differential assessment of which was not possible under the conditions of this study. According to the results, the relative number of phagocytic microglia (phagocytic index, (PI)) in animal models of AD was 2 times higher than in intact animals. It should be noted that this value was significantly lower in sham-operated (SO) animals. Endocytic activity (PI) in AD rats was also significantly (almost 5-fold) higher than in intact animals and 2 times higher than that in sham-operated rats (FIG. 10A and FIG. 10B). Administration of the test substance caused complete normalization of both the relative number of phagocytic microglia and the levels of their endocytic (phagocytic) activity, which indicates anti-inflammatory effect of the drug candidate.

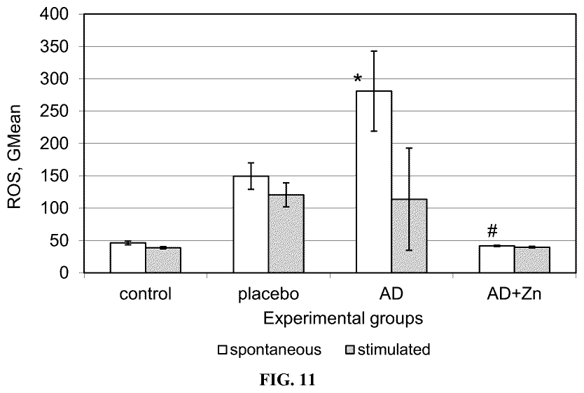

Oxidative metabolism is another metabolic indicator of microglia. In this study, microglia in intact animals were characterized by the absence of reaction to in vitro stimulation by bacterial LPS, which indicates their involvement in the inflammaging associated with aging (FIG. 11). Norden D M, Godbout J P. Review: microglia of the aged brain: primed to be activated and resistant to regulation. Neuropathol Appl Neurobiol. 2013; 39(1):19-34. doi:10.1111/j.1365-2990.2012.01306.x.

Oxidative metabolism in sham-operated animals was somewhat enhanced compared with intact animals at the time of the experiment, which supports the assumption about a persistent reparative inflammatory process aggravated by inflammaging. An additional criterion for this condition is the lack of a functional reserve of oxidative metabolism in microglia in animals of this group in response to in vitro LPS treatment.

AD progression was accompanied by a significant (5-fold) increase in the generation of reactive oxygen species by microglial cells. Enhanced oxidative metabolism in microglia is an essential component of neuroinflammation associated with AD, therefore, the data support the validity of the selected model. The reaction to in vitro LPS treatment of microglial cells in animals of this group was sharply negative, which indicates an extreme degree of their pro-inflammatory activation. Administration of the test substance resulted in a complete normalization of oxidative metabolism in microglia in AD rat models: both the basal level of ROS generation and the metabolic reserve of this function. Thus, an analysis of the metabolic values of the microglial functional polarization showed the presence of a pro-inflammatory metabolic shift in AD rat models and its elimination after a therapeutic course of treatment with the test substance.

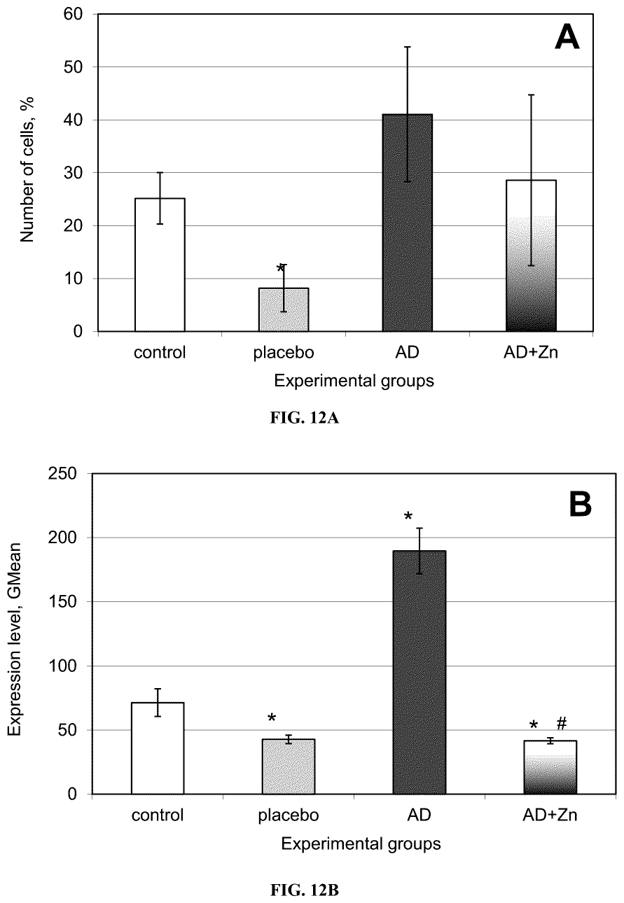

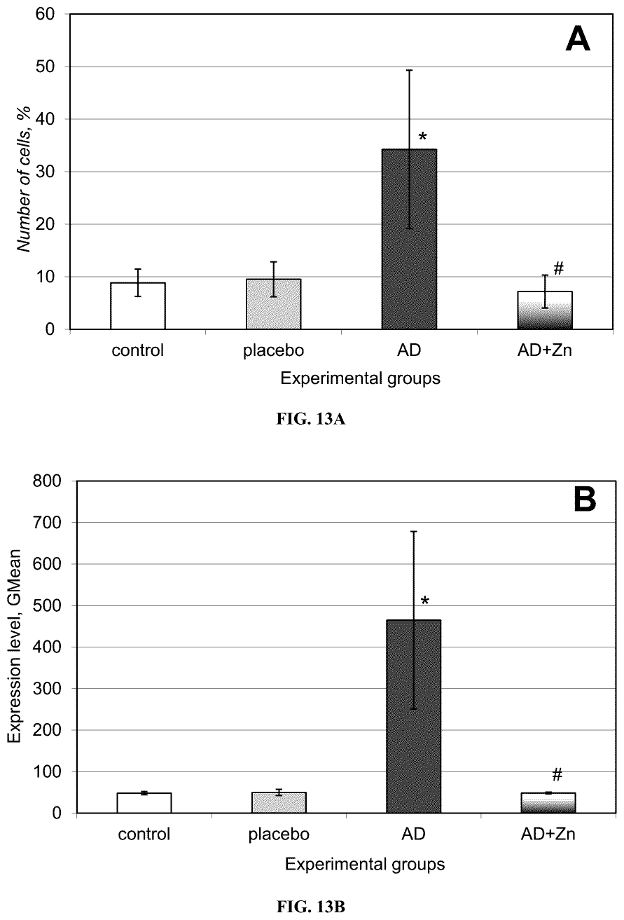

To characterize the phenotypic profile of microglia, the following markers were used: CD206 (scavenger receptor, a marker of alternative polarization of phagocytes of extra-cerebral localization and also a marker of activated resident microglia) and CD86 (a co-stimulatory molecule involved in the process of antigen presentation, a marker of pro-inflammatory activation of phagocytes of extra-cerebral localization and which is also overexpressed by myeloid-derived suppressor cells, negative regulators of proinflammatory reactions of innate and adaptive immunity). There was significant variability in the quantitative analysis of phenotypic microglial markers, probably due to uneven aging processes and a small statistical sampling of animals. In general, the results of assessment of the phenotypic profile of microglial cells are shown in FIG. 12A, FIG. 12B, FIG. 13A and FIG. 13B.

The number of CD86+ cells in the microglia population of animal models of AD was 1.6 times higher compared with intact animals (FIG. 12A and FIG. 12B). The expression level of this marker in microglial cells was more than 2.5 times higher than in intact animals, which evidences a pro-inflammatory shift in the microglial functions characteristic of AD and confirms the results of assessment of the metabolic parameters of these cells. Administration of the test substance normalized both the number of CD86+ cells and the expression level of this marker in positive cells, which indicates a powerful anti-inflammatory effect of the drug candidate.

The data on CD86 expression are also supported by the data on expression of another phenotypic marker, CD206 (FIG. 13A and FIG. 13B).

The number of microglial cells expressing CD206 in AD rat models was 3.5 times higher than that of intact animals, which indicates phagocytic activation in the brains of AD rats. The expression level of this marker in positive cells in AD rats was more than 5 times higher than that of intact rats. Treatment with the zinc-based test substance caused a decrease in the above values to the levels of intact animals, which is another evidence of anti-inflammatory effect of the test substance.

Thus, the progression of Aβ1-40-induced AD is accompanied by a pronounced pro-inflammatory functional shift of microglial cells. The use of ⁶⁴Zn-asp as monotherapy normalizes phenotypic and functional parameters of microglia: all the analyzed characteristics of this phagocyte population at the time of the experiment did not differ from the parameters in healthy animals of the corresponding age group.

### Therapeutic Effects of ⁶⁴Zn-Asp on Blood Count Values in Rat Models of Alzheimer's Disease Induced by the Infusion of Aβ1-40

Literature data provide strong evidence that chronic inflammation is one of the most important pathophysiological components of synucleinopathies and taupathias, including AD. Leukograms of patients with AD reveal an increased number of monocytes and neutrophils and a low lymphocyte count. Escalated levels of monocytes and neutrophils are hallmarks of chronic inflammation and may be both precursor to AD and its consequence. A low number of lymphocytes specifies that the body's resistance to the fight infection is significantly reduced. Shad K F et al., Synapse. 2013; 67(8):541-543. doi:10.1002/syn.21651; Stock A J, Kasus-Jacobi A, Pereira H A. J Neuroinflammation. 2018; 15(1):240 doi:10.1186/s12974-018-1284-4. Increased permeability of the blood brain barrier (BBB) in AD facilitates migration of neuroinflammatory mediators to the periphery and recruitment of circulating leukocytes to the brain, which creates prerequisites for the persistent meta-inflammatory process. Yamazaki Y, Kanekiyo T. Int J Mol Sci. 2017; 18(9):1965 doi:10.3390/ijms1809196. Blood count values were measured in the experimental animals at the end of the experiment.

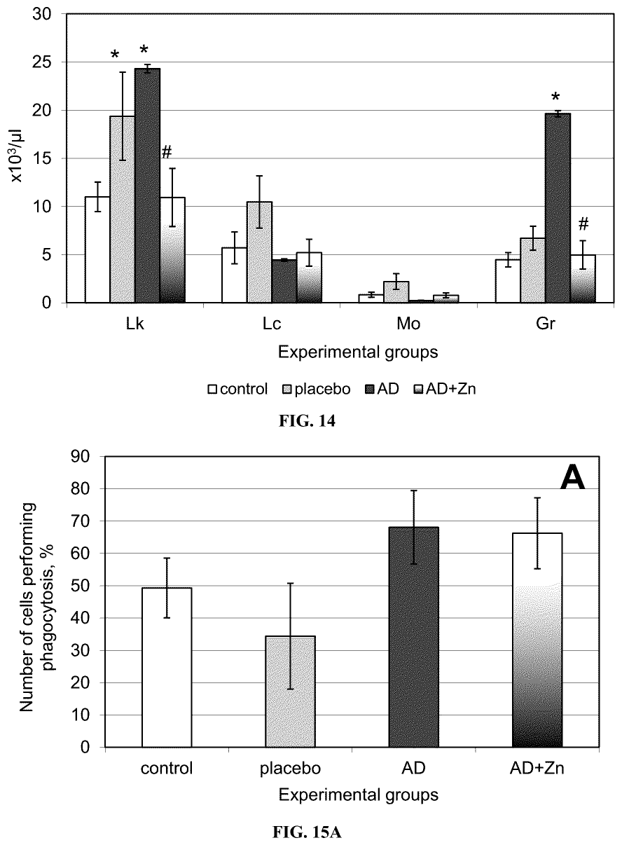

Analysis of blood samples from rat models of Aβ1-40-induced AD showed extremely high white blood cell (WBC) counts: the number of circulating leukocytes in their blood was 2.5 times higher compared with intact animals (FIG. 14). It should be noted that leukocytosis was also observed in the placebo-treated rats, which is apparently due to the low reparative potential of the immune system in old animals. Treatment with the test substance resulted in a complete normalization of the absolute number of circulating leukocytes in animal models of AD.

Analysis of the population composition of circulating leukocytes showed a slight decrease in the lymphocyte count and a significant decrease in the monocyte count (moderate monocytopenia). AD induction was also accompanied by impressive neutrophilia with a significant (more than 4-fold) increase in the neutrophil-lymphocyte ratio (the ratio of absolute neutrophil count to absolute lymphocyte count in peripheral blood, NLR). NLR is one of the early markers of AD progression (Kuyumcu M E et al., Dement Geriatr Cogn Disord. 2012; 34(2):69-74. doi:10.1159/000341583) and an important biomarker for the identification of patients with cognitive impairment. Dong X et al., Front Aging Neurosci. 2019; 11:332 Published 2019 Dec. 5. doi:10.3389/fnagi.2019.00332. Administration of the zinc-based preparation caused complete normalization of NLR, both due to an increase in the lymphocyte count (which is a criterion for inflammation the resolution by activating the suppression function of regulatory cells) and due to a significant decrease in the segmented neutrophil count.

### Therapeutic Effects of ⁶⁴Zn-Asp on Functional and Phenotypic Properties of Circulating Phagocytes in Rat Models of Alzheimer's Disease Induced by the Infusion of Aβ1-40

As described above, the development of AD is accompanied by the formation of systemic inflammation which increases and maintains the persistence of neuro-inflammatory processes. This circumstance makes effector cells of the systemic inflammatory process no less attractive targets for anti-inflammatory therapy in AD than resident leukocytes. This was one of the reasons for analyzing functional and phenotypic properties of circulating phagocytes in rat models of Aβ1-40-induced AD. In addition, the test substance was administered intravenously, which makes circulating phagocytes the first line of respondent cells. As mentioned above, the results of blood counts showed the presence of a systemic inflammatory process in rat models of Aβ1-40-induced AD with significant leukocytosis, neutrophilia and increased neutrophil-lymphocyte ratio, a validated biomarker of systemic inflammatory process in the progressive form of AD. Analysis of the functional and phenotypic properties of circulating phagocytes confirmed these observations.

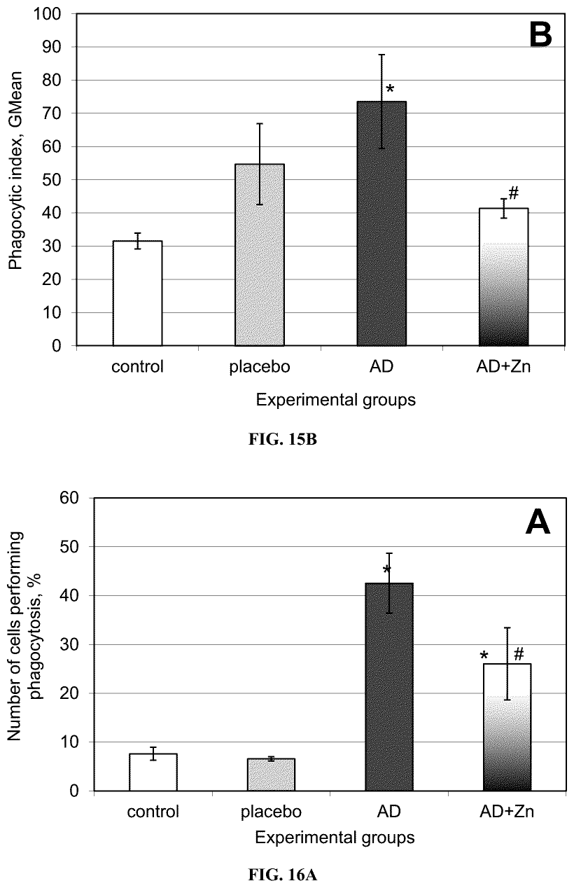

Neutrophilia detected in the blood samples from AD rats was accompanied by a significant increase in the phagocytic activity, which is, on the one hand, a marker of an active state of the cells, and on the other hand, a sign of the anti-inflammatory shift in their metabolism (FIG. 15B). Moreover, the relative number of phagocytic neutrophils in animals of this group did not differ significantly from that in intact and SO rats (FIG. 15A). It should be noted that the absorption activity of polymorphonuclear phagocytes in SO rats was significantly higher than in the intact controls, which may be a consequence of surgery and the associated activation of repair processes, characterized by an anti-inflammatory shift in phagocytic cell metabolism.

Treatment with the test substance was accompanied by a decrease in the phagocytic activity of these cells virtually to the values shown by the intact animals which indicates its homeostatic systemic effect.

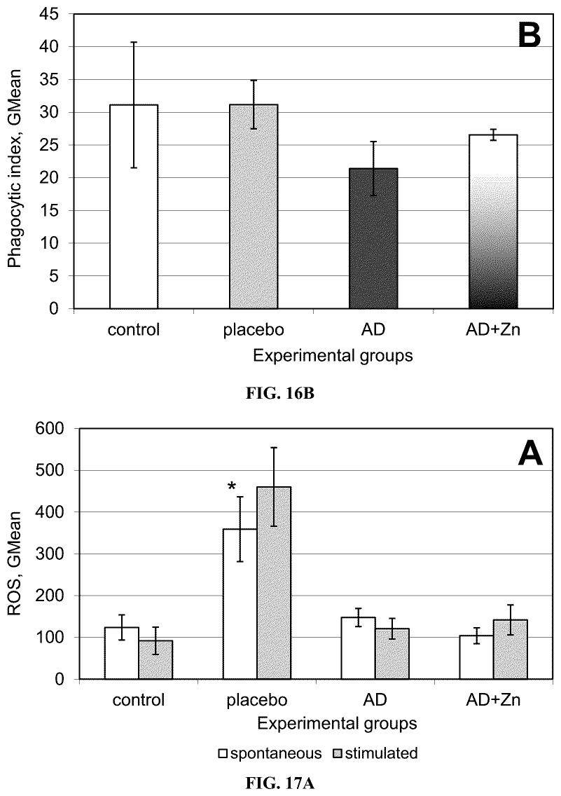

The relative number of monocytes performing phagocytosis in rat models of AD was almost four times higher compared with the intact and SO rats (FIG. 16A). Moreover, the phagocytic activity of these cells did not differ from that in both control groups of animals (FIG. 16B).

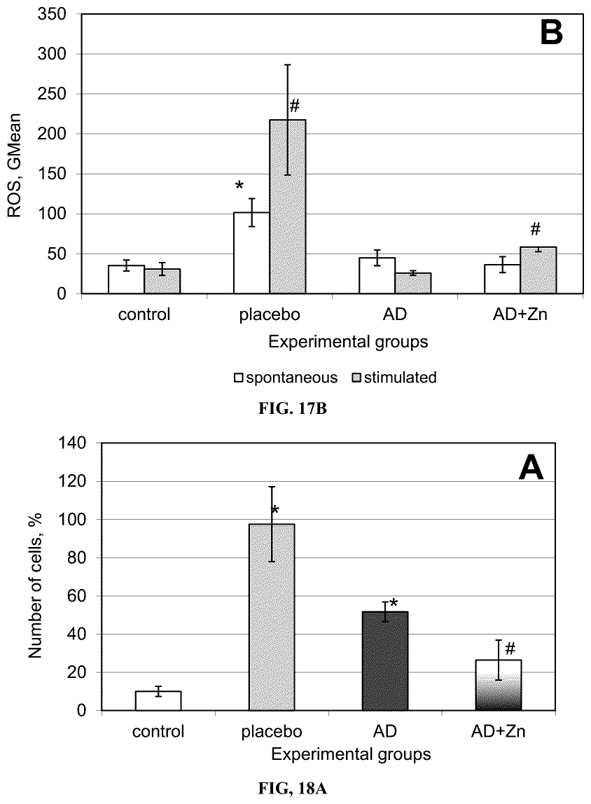

The analysis of indices of oxidative metabolism in circulating phagocytes of both populations did not reveal any statistically significant differences between AD animal models and intact animals (FIG. 17A and FIG. 17B). It should be noted that administration of the zinc-based preparation to AD rats caused the formation of a small functional reserve of oxidative metabolism in the analyzed populations of circulating phagocytes, which may be a sign of the presence of “young” cells in the peripheral blood and, therefore, indicate the ability of the drug candidate to moderately stimulate myelopoiesis.

Sharply increased indices of oxidative metabolism both in granulocytes and mononuclear phagocytes of the peripheral blood were recorded in sham-operated animals. At the same time, there was a functional reserve of oxidative metabolism. In all likelihood, this picture may reflect persistent reparative inflammation with activation of medullary myelopoiesis.

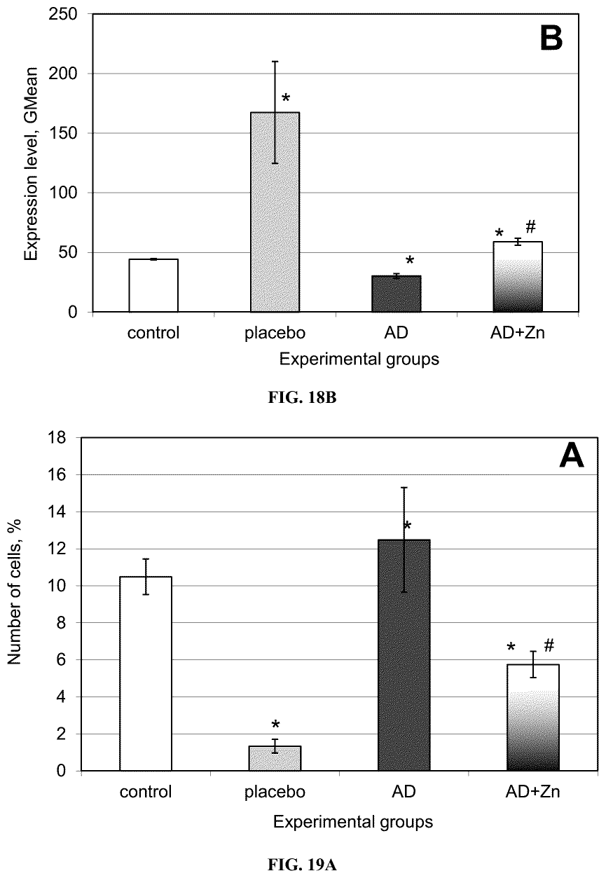

Analysis of phenotypic markers of circulating phagocytes in animal models of AD also confirms spontaneous resolution of systemic inflammation. The relative number of CD86+ circulating phagocytes in AD rats is significantly higher than in control animals (FIG. 18A and FIG. 18B).

As mentioned above, this marker is characteristic of both phagocytes with a pro-inflammatory metabolic shift and myeloid suppressor cells. Taking into account the increased phagocytic activity of peripheral blood phagocytes, it can be assumed that an increase in the fraction of CD86+ cells was due to the presence of myeloid suppressor cells.

An increased fraction of CD86+ cells with an increased level of expression of this marker was found in sham-operated (SO) animals, which complements the picture of a reparative process induced by the surgical procedure.

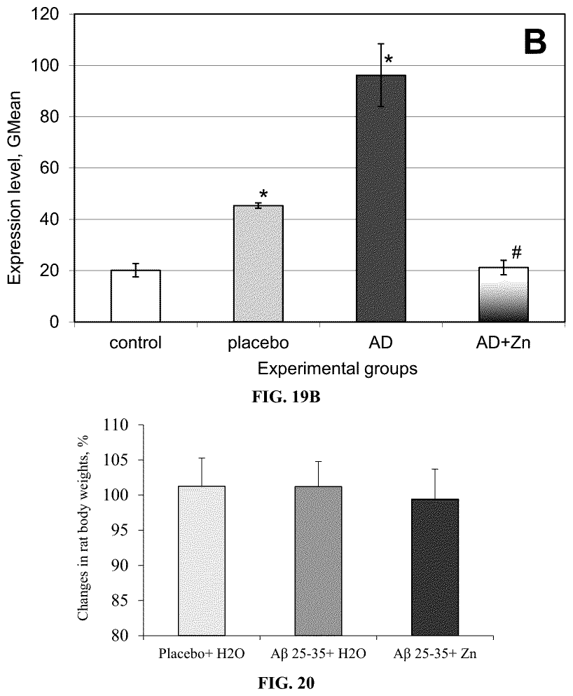

Analysis of the CD206 expression also supports the resolution of inflammation. In AD rats, the fraction of CD206 marker positive cells did not differ in size from that in intact animals. However, its expression level by circulating phagocytes was higher than in the intact control (FIG. 19A and FIG. 19B).

Administration of the test substance as monotherapy brought the number of cells expressing these markers and their expression levels to normal, which confirms its homeostatic effect on systemic immune reactivity in the progression of AD induced by Aβ1-40 infusion.

### Findings

A decrease in the body weight and a decrease in water and feed intake in rat models of Aβ1-40-induced AD was observed 3 weeks after mimicking the disease. These parameters were restored in AD rats treated with ⁶⁴Zn-asp for 10 days.

A decreased number of hippocampal dopaminergic neurons and decreased expression of tyrosine hydroxylase (TH) in hippocampal dopaminergic neurons were recorded in rat models of Aβ1-40-induced AD. Administration of ⁶⁴Zn-asp to Aβ1-40 AD rats increased the staining intensity of TH-immuno-positive cells rather than their number.

Progression of Aβ1-40-induced AD was associated with impairment of cognitive flexibility in AD rats, which indicates impaired function of the frontal cortex. Rat models of Aβ1-40-induced Alzheimer's disease did not exhibit any changes in their ability to spatial learning or short-term/long-term memories (hippocampal function). Administration of ⁶⁴Zn-asp significantly improved the cognitive function in AD models and virtually returned it to the values in intact and sham-operated animals.

Progression of Aβ1-40-induced AD was characterized by a prolonged acute local (in microglia) inflammatory process and a moderately expressed systemic inflammation with signs of its spontaneous resolution.

Therapy with the zinc-based test substance resulted in an almost complete resolution of neuroinflammation and homeostatic regulation of systemic immune reactivity, which indicates a pathogenetic nature of its therapeutic effect.

### Results of the Study II

### Therapeutic Effects of ⁶⁴Zn-Asp on Cognitive Activity as Well as Local and Systemic Immune Reactivity in Rat Models of Alzheimer's Disease Induced by Infusion of Aβ25-35

### Effects of ⁶⁴Zn-Asp on Cognitive Symptoms in Rat Models of Aβ25-35-Induced Alzheimer's Disease

### Effects of ⁶⁴Zn-Asp on Body Weight Changes in Rat Models of Aβ25-35-Induced Alzheimer's Disease

Animal body weight is a classic clinical sign that characterizes the general condition of an animal, and its loss during the experiment indicates that the condition of the animal is deteriorating. No significant changes were observed in the body weight of rat models of Aβ25-35-induced AD during one month of the experiment (FIG. 20).

Administration of ⁶⁴Zn-asp had no effect on this parameter.

### Effects of ⁶⁴Zn-Asp on Tyrosine Hydroxylase Expression in the Hippocampus of Rat Models of Aβ25-35-Induced Alzheimer's Disease

According to the result of immunohistochemical analysis for the expression of tyrosine hydroxylase the quick score (Q) in AD rat models was 6.0±0.0. The staining intensity of TH-positive cells in sham-operated animals did not differ significantly from that of intact rats and was 7.0±1.7 scores (Table 5).

TABLE 5
Staining intensity and percentage of TH-positive hippocampal cells in
rat models of Aβ25-35-induced Alzheimer's disease

| | | Assessment | Assessment | |
|---|---|---|---|---|
| | Animal | (positive | (intensity | |
| | number | cells, P) | of staining, I) | Q = P × I |
| Intact | 658 E | 3 | 2 | 6 |
| animals | 658 F | 3 | 2 | 6 |
| | 658G | 3 | 2 | 6 |
| Placebo | 659 A | 3 | 2 | 6 |
| (H₂O) | 659 B | 3 | 3 | 9 |
| | 659 C | 3 | 2 | 6 |
| Aβ 25-35 + | 662 A | 3 | 2 | 6 |
| H₂O | 662 B | 3 | 2 | 6 |
| | 662 C | 2 | 2 | 4 |
| Aβ 25-35 + | 663 A | 3 | 2 | 6 |
| Zn | 663 B | 2 | 2 | 4 |

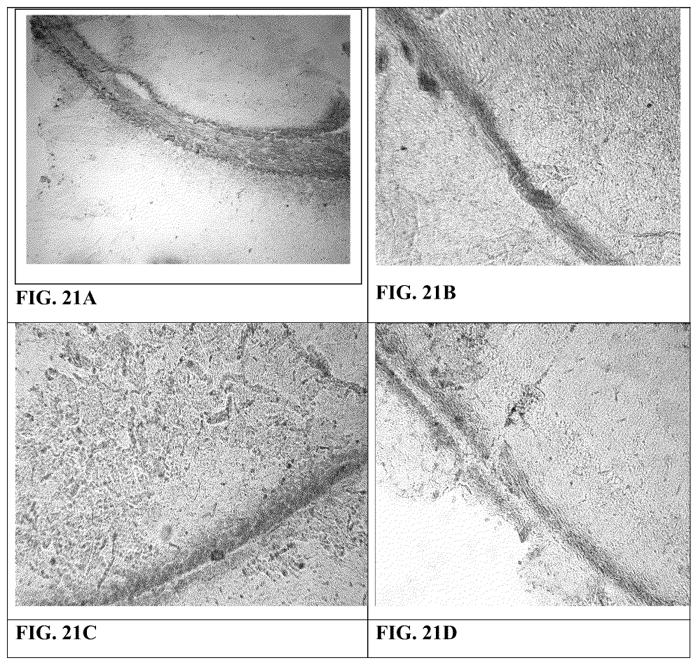

In animal models of Aβ25-35-induced AD, there were no statistically significant changes either in the number of TH-positive neurons or the intensity of staining compared with the intact and placebo-treated animals (Q=5.3±1.5). Administration of ⁶⁴Zn-asp to rat models of Aβ25-35-induced AD had no effect on the staining intensity of TH-positive cells; the Q value in this group was 5.0±1.4 (FIG. 21A, FIG. 21B, FIG. 21C, and FIG. 21D).

The analysis did not reveal any significant changes in the function or number of TH-positive hippocampal neurons.

### Effects of ⁶⁴Zn-Asp on the Spatial Memory in Rat Models of Aβ25-35-Induced Alzheimer's Disease

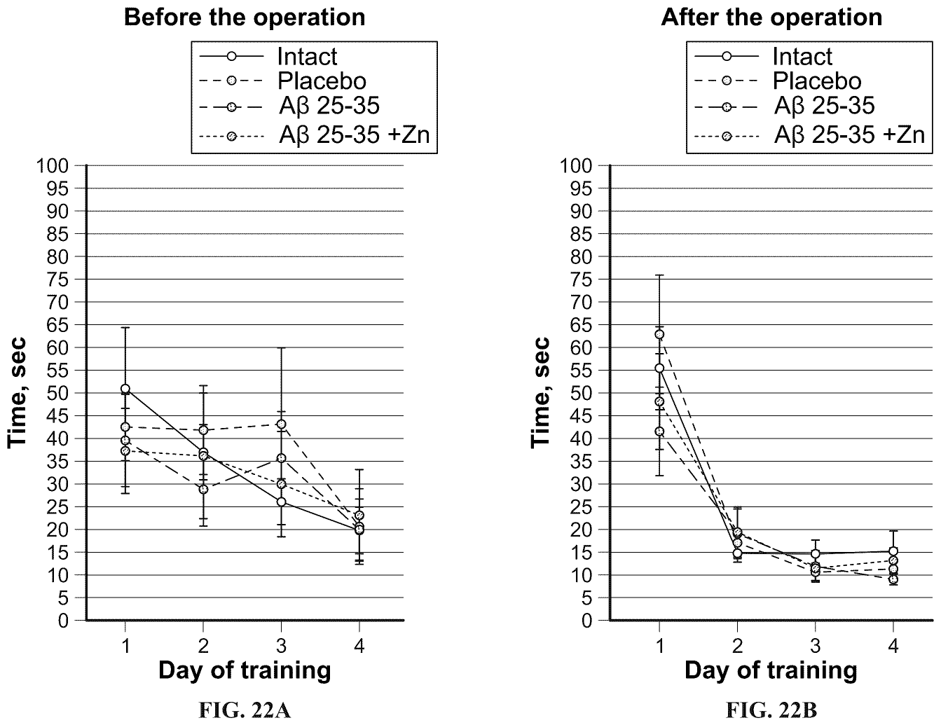

To assess the time spent on spatial learning in Alzheimer's disease, the time spent to find the “escape box” during 4 days of training before the operation and during 4 days of training after the operation (starting from day 18 after the operation) was compared. Different locations were used of the “escape box” in the training and probe phases before and after surgery. As can be seen in FIG. 22A, and FIG. 22B, during 4-day trainings, both before and after surgery, all groups of rats reduced the time spent to find an entrance to the escape hole. No difference in time was found between the control groups (intact and placebo-treated animals) and rat models of Aβ25-35-induced Alzheimer's disease.

To assess short-term memory, 24 hours after the last 4-day training phase (on the 5^{th} day after the start of training), the rats were placed in the Barnes maze, but the entrance to the “escape box” was closed. To assess long-term memory, the same test was repeated on the 5^{th} day following the 4-day training phase.

Time spent by each animal to find the “escape box” was measured. The faster the animal found the correct hole, the higher was the level of its spatial memory (the hippocampal function). The level of cognitive flexibility was also assessed by measuring time the animal spent near the entrance to the escape hole—the less time the animal was there, the higher level of cognitive flexibility it possessed (the frontal cortex function), i.e., the animal realized faster that the escape hole should be sought elsewhere.

Displayed in Tables 6 and 7, the values shown by animals from all groups before the operation were rather individual, therefore it was logical to compare patterns of changes rather than absolute numbers.

As displayed in Table 6, the time rats from all groups spent searching for the “escape box” before the operation naturally increased between the tests for this parameter 24 hours and 5 days after the probe phase. Only rats that were later infused Aβ25-35 to mimic AD were observed to reduce the time spent to find the “escape box”, which may be associated with individual traits of these rats.

TABLE 6
Effects of ⁶⁴Zn-asp on the spatial short-term and long-term memory
efficiency (time spent to find an entrance to the “escape box”) in rat models of
Aβ25-35-induced Alzheimer's disease, M ± SD

| | Short-term memory | | | Long-term memory | | |
|---|---|---|---|---|---|---|
| | 24 hours following training | | | 5 days following training | | |
| | Time spent to find the | | | Time spent to find the | | |
| | “escape box”, sec | | | “escape box”, sec | | |
| | before | after | % of | before | after | % of |
| Groups | surgery | surgery | changes | surgery | surgery | changes |
| Intact rats, | 12.14 ± 4.03 | 5.71 ± 1.25 | −52.94 | 26.57 ± 6.5 | 9.86 ± 4.29 | −62.9 |
| n = 7 | | | (p = 0.37) | | | (p = 0.13) |
| Sham-operated | 5.5 ± 1.31 | 4.5 ± 1.06 | −18.18 | 24.33 ± 9.17 | 25.17 ± 13.35 | n/s |
| rats, n = 7 | | | | | | |
| Aβ 25-35 + H₂O, | 10.43 ± 2.69 | 11.43 ± 4.29 | n/s | 4.57 ± 0.75 | 10.2 ± 3.73 | 125 |
| n = 7 | | | | | | (p = 0.45) |
| Aβ 25-35 + ⁶⁴Zn- | 13.33 ± 7.8 | 8.33 ± 5.76 | −37.5 | 14.67 ± 3.53 | 21.83 ± 12.53 | 48.86 |
| asp, n = 7 | | | (p = 0.37) | | | (p = 0.68) |

During the probe phase (18 days after surgery), time spent to find the “escaoe box” by rats of all experimental groups in the short-memory test was either slightly reduced or left the same. When the animals were tested for the long-term memory, the picture was virtually similar in the intact and sham-operated rats, while rat models of Aβ25-35-induced AD were observed to increase time spent to find the correct hole. The same pattern was observed in the group of rat models of Aβ25-35-induced AD treated with ⁶⁴Zn-asp. However, the changes observed in both groups were not statistically significant. It can therefore be concluded that there is no statistically significant impairment in short-term memory in rat models of Aβ25-35-induced Alzheimer's disease but there is a tendency towards impairment of long-term memory that was not improved by ⁶⁴Zn-asp.

When assessing cognitive flexibility in rats 24 hours and 5 days after a 4-day probe phase by measuring time the animal spent near the entrance to the “escape box”, either natural decrease or no changes in this parameter (Table 7) was observed.

TABLE 7
Effects of ⁶⁴Zn-asp on the cognitive flexibility in rat models of
Aβ25-35-induced Alzheimer's disease 24 hours and 5 days after the last training trial, M ± SD

| | Short-term memory | | | Long-term memory | | |
|---|---|---|---|---|---|---|
| | 24 hours following training | | | 5 days following training | | |
| | Time spent near the | | | Time spent near the | | |
| | entrance to the “escape | | | entrance to the “escape | | |
| | box”, sec | | | box”, sec | | |
| | before | after | % of | before | after | % of |
| Groups | surgery | surgery | changes | surgery | surgery | changes |
| Intact rats, | 23 ± 2.53 | 18 ± 2.18 | −21.74 | 19.14 ± 1.94 | 16.29 ± 2.31 | 14.93 |
| n = 7 | | | (p = 0.04) | | | (p = 0.31) |
| Sham-operated | 29.33 ± 2.55 | 21.83 ± 6.67 | −25.57 | 28 ± 5.3 | 25 ± 6.31 | −10.71 |
| rats, n = 7 | | | (p = 0.22) | | | (p = 0.78) |
| Aβ 25-35 + H₂O, | 23.43 ± 2.78 | 17.43 ± 1.88 | −25.61 | 41.86 ± 5.12 | 28.43 ± 5.4 | −32.08 |
| n = 7 | | | (p = 0.21) | | | (p = 0.01) |
| Aβ 25-35 + ⁶⁴Zn- | 27.33 ± 4.06 | 30.67 ± 7.32 | 12.2 | 30.67 ± 4.32 | 18.17 ± 5.38 | −40.76 |
| asp, n = 7 | | | (p = 0.74) | | | (p = 0.13) |

This pattern was characteristic of all groups of animals before the operation, except for the rats, which were subsequently used to model Alzheimer's disease, who showed an increase in time spent near the escape hole, which may be associated with the individual traits of these rats. During the probe phase, intact and sham-operated animals as well as Aβ25-35 AD rats were observed to spend a little less time near the entrance to the “escape box”. This indicates a normal level of cognitive flexibility in AD rats and the absence of influence of Aβ25-35 on this parameter. No significant changes in this parameter after the test substance therapy were observed either.

Therapeutic Effects of ⁶⁴Zn-Asp on the Aβ and Tau Protein Levels in Hippocampal Homogenate from Rat Models of Alzheimer's Disease Induced by the Infusion of Aβ25-35

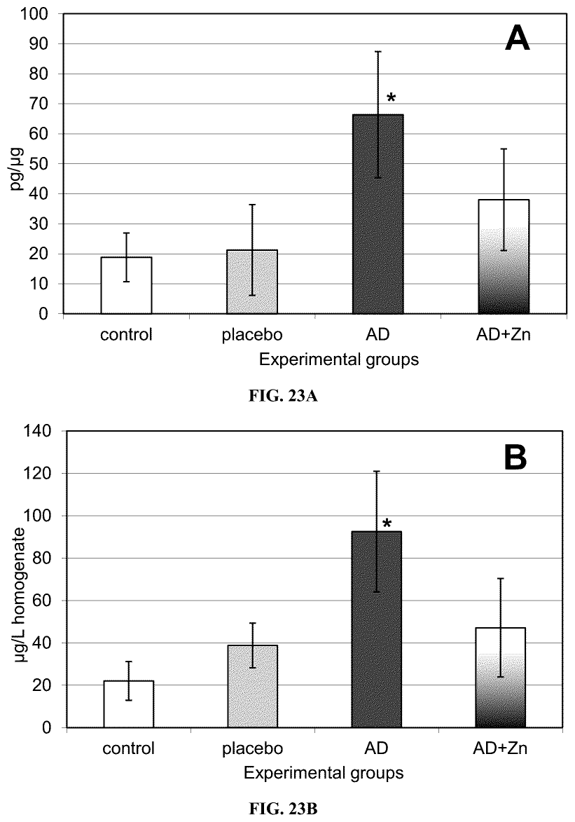

The analysis of the levels of proteins involved in the pathophysiological processes in AD (Aβ and tau protein) in hippocampal homogenates from Aβ25-35 AD rat models showed results similar to those obtained from animal models of AD induced by Aβ1-40 infusion. The levels of both Aβ and tau protein in AD controls significantly exceeded the values in intact and SO rats (FIG. 23A, and FIG. 23B). Therapeutic treatment with the test substance resulted in a significant decrease in the levels of these proteins but did not bring them back to normal completely. It should be noted that, as with the models of AD induced by Aβ1-40 infusion, the values in both proteins in the hippocampal homogenates were characterized by a very high degree of variability, which makes the results inconclusive. Such variability in values may be caused by the following factors:

method of sample preparation recommended by the manufacturer of test systems used in the study differs from those described in protocols for almost all test systems used for the same purpose and presented in the literature;
small number of animals in all experimental groups used to analyze this parameter (given the fact that the study was declared as pilot).

However, the results of analysis of these parameters suggest that the test substance has a pathogenetic therapeutic effect accompanied by a decrease in the quantitative characteristics of the pathogenetic markers of Alzheimer's disease.

### Therapeutic Effects of ⁶⁴Zn-Asp on Functional and Phenotypic Properties of Microglia in Rat Models of Alzheimer's Disease Induced by the Infusion of Aβ25-35

As stated above, the model of AD induced by Aβ25-35 infusion was chosen because of an exceptional role of Aβ fragment in the formation of senile plaques in AD, its ability to have a direct toxic effect on neurons leading to their death regardless of the formation of Aβ deposits, as well as the fact of development of autoimmune reactions directed against this peptide and accompanying the progression of AD. Analysis of the functional and phenotypic characteristics of microglia in rat models of AD induced by Aβ25-35 infusion showed the following.

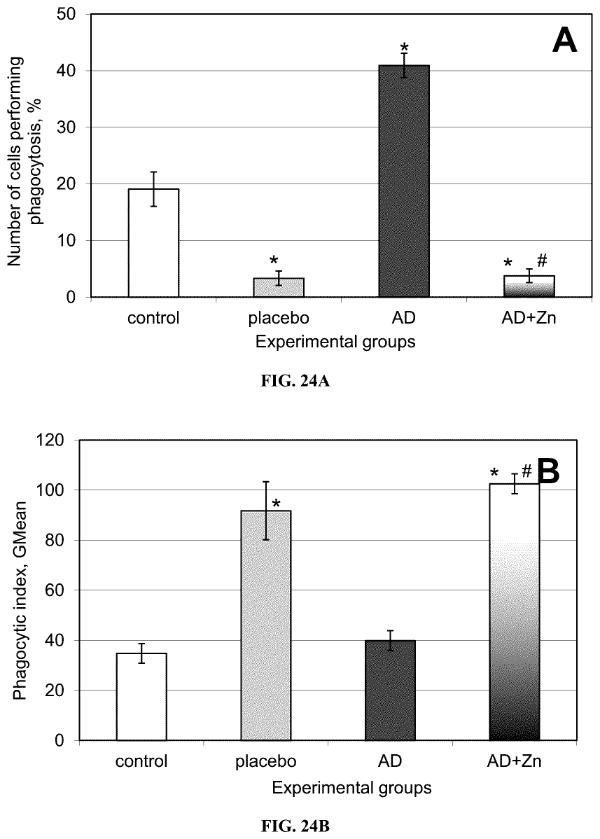

The number of microglial cells performing phagocytosis in control AD rats was more than 2 times as high as in the intact and SO animals, which indicates an activated state of a complex population of phagocytes in the brain (FIG. 24A, and FIG. 24B). However, the intensity of absorption activity of microglia in animal models of AD did not differ from that in intact animals. Consequently, an increase in the absorption activity could result from the anti-inflammatory activation of microglia caused by spontaneous resolution of neuroinflammation (since the analysis was carried out at the end of the experiment).

It should be noted that a high level of absorption activity of microglia in SO animals was observed, which was probably associated with reparative processes after surgery. Therapy with the test substance caused a sharp decrease in the number of phagocytic microglial cells in AD rat models: 10-fold compared with AD controls and 5-fold compared with intact animals. At the same time, the rate of phagocytosis increased significantly. Any changes in phagocyte function, regardless of their location, should be analyzed in the context of changes in other metabolic reactions. In this case, one should probably take into account the fact that AD modeling (including sham surgery) leads to changes in BBB permeability and migration of circulating phagocytes to microglia. As a result of these processes, the microglia population includes, in addition to resident macrophages, recruited mononuclear and polymorphononuclear phagocytes, differential assessment of which was not provided by the terms and conditions of the study. Considering the above, the data on phagocytic activity can be interpreted as signs of stimulation of reparative processes by the test substance, since an increase in absorption activity of microglia is characteristic of extracerebral phagocytes (the proportion of which in the complex microglia population may be quite significant) of the anti-inflammatory phenotype.

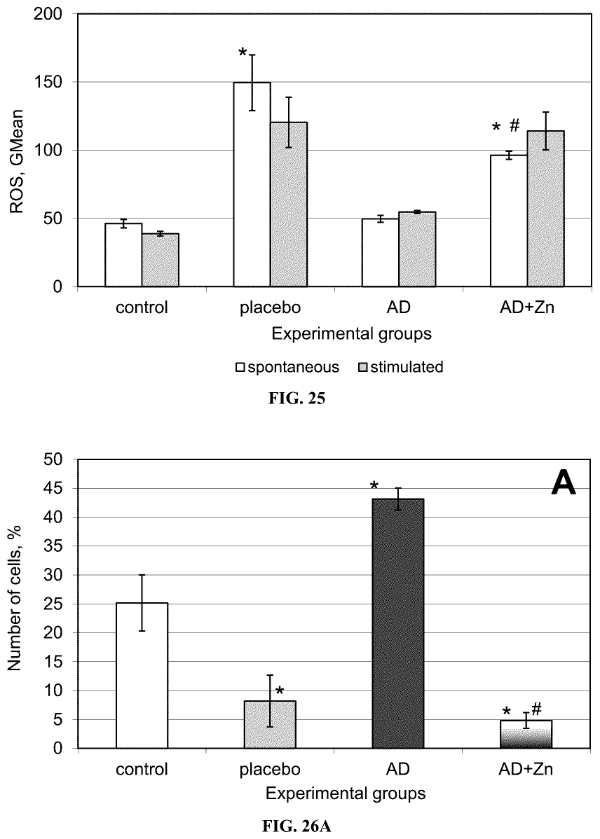

The results of assessment of the phagocytic activity of microglia in animal models of AD induced by the infusion of Aβ25-35 were supported by the results of assessment of the oxidative metabolism in these cells (FIG. 25).

The levels of ROS generation in rat models of Aβ25-35 AD did not differ from those in intact animals and were significantly lower than in sham-operated rats. Assessment of the activated state of microglia in sham-operated rats as a marker of a persistent reparative process caused by surgical intervention is validated by the analysis of physiological state of animals of this group, which was absolutely satisfactory, without deviations in their cognitive activity or behavioral reactions. Consequently, increased levels of ROS generation in SO animals may be considered as an indicant of the reparative inflammatory process. The absence of differences between the oxidative metabolism in microglia in the rat models of Aβ25-35-induced AD and intact animals may indicate a spontaneous resolution of neuroinflammation and the imperfection of the AD model used in the study. Administration of the test substance to rat models of Aβ25-35-induced AD increased oxidative metabolism in their microglia, which may be evidence of stimulation of the repair processes by the drug candidate.

The expression levels of phenotypic markers of microglial cells are generally consistent with their metabolic profile (FIG. 26A and FIG. 26B), however, they contain an element requiring a more detailed study of microglia in this AD model.

The number of CD86+ cells in the microglia population in AD animals was significantly higher than in intact animals. If we consider CD86+ cells as a marker of myeloid suppressor cells, then the data obtained are consistent with the concept of spontaneous regression of neuroinflammation in AD rat models, which indicates the imperfection of the model. However, if one attributes an increase in the fraction of CD86+ cells to the elevated number of effector phagocytes activated in antigen presentation, then the results of the analysis of phenotypic markers indicate activation of intracerebral autoimmune reactions initiated by the infusion of Aβ25-35, which is consistent with literature data on the participation of Aβ25-35 in the autoimmune component of AD. In this case, sharp reduction in the fraction of CD86+ cells in AD animals after a course of therapy with the zinc-based preparation can be considered as evidence of the ability of the test substance to inhibit the development of autoimmune reactions associated with AD.

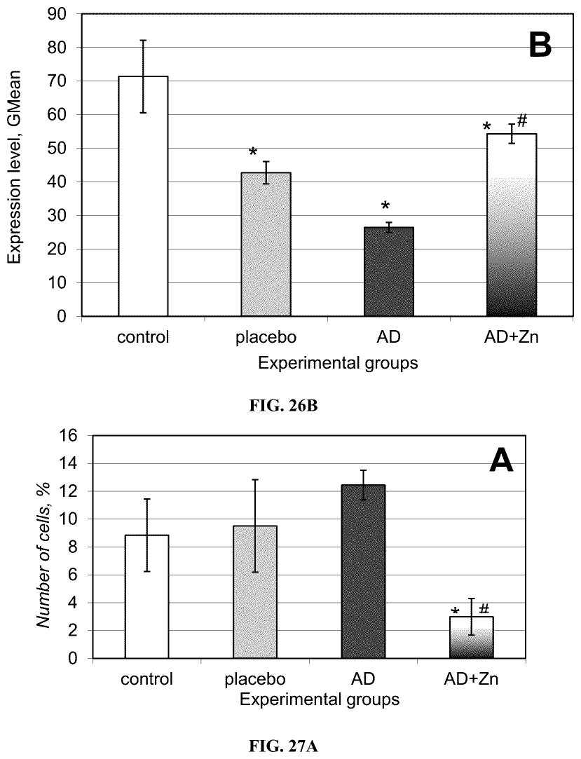

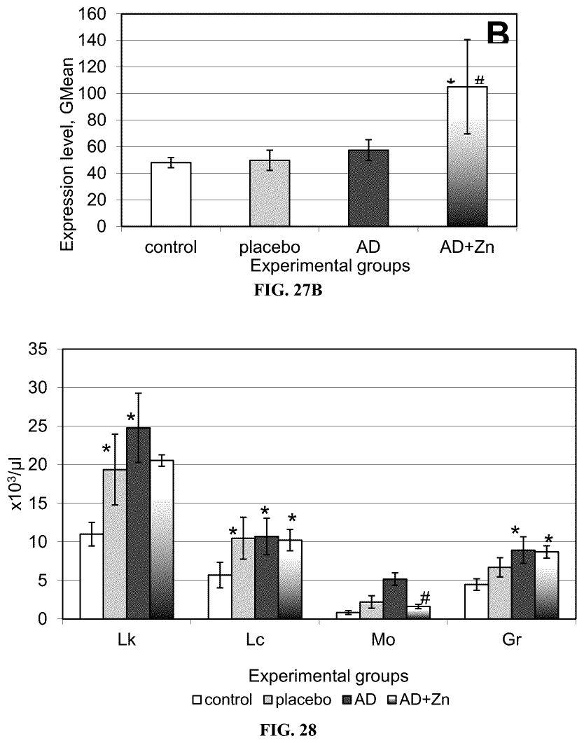

This assumption does not contradict the results of the assessment of the expression of CD206, another microglia phenotype marker (FIG. 27A and FIG. 27B).

The levels of expression of this marker in microglia and the size of the fraction of positive cells in AD animals did not significantly differ from the compared values in intact animals. If one considers that this marker indicates an activated state of microglia, then the downsized fraction of positive cells as a result of the test substance action can be considered evidence of its homeostatic therapeutic effect.

### Therapeutic Effects of ⁶⁴Zn-Asp on Functional and Phenotypic Properties of Circulating Phagocytes in Rat Models of Alzheimer's Disease Induced by the Infusion of Aβ25-35

Differential blood counts of rat models of Aβ25-35-induced AD showed even more pronounced inflammation than those of Aβ1-40 AD models (FIG. 28) but had somewhat different features.

The number of circulating leukocytes in AD rat models was twice as high as in intact animals, lymphocytes and neutrophilic granulocytes also doubled in number, and the number of monocytes increased almost 4-fold compared with intact animals. At the same time, the neutrophil-lymphocyte ratio (NLR) in AD rats was significantly lower than in intact animals. Such an increase in the number of lymphocytes can be evidence of the activation of self-reactive T-cell immune responses (autoimmunity), which is consistent with the proposed interpretation of the results of assessment of the functional and phenotypic profile of microglia. A therapeutic course with the test substance caused a slight decrease in leukocytosis but did not bring the leukocyte counts back to normal. This decrease was mainly due to normalization of the number of monocytes. However, the levels of neutrophils and lymphocytes after the treatment were unchanged and the NLR remained as high as in untreated AD rats.

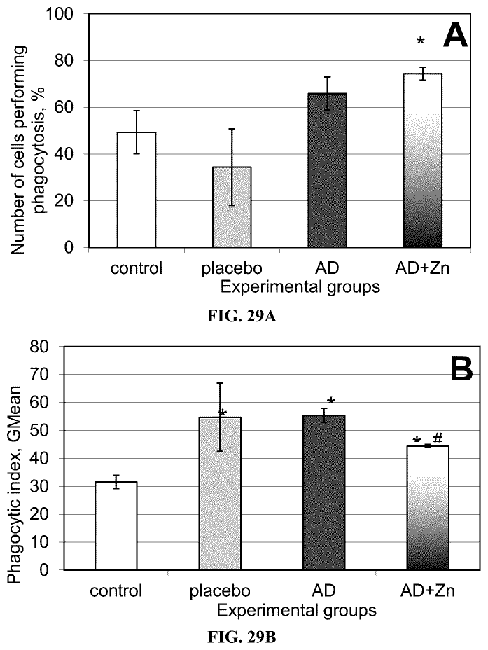

The relative number of neutrophils performing phagocytosis in the peripheral blood of rat models of AD was higher than in intact animals. However, the difference was insignificant (FIG. 29A and FIG. 29B).

The rate of phagocytosis by circulating polymorphonuclear phagocytes in rat models of AD was significantly higher than in control animals, which is a sign of the activated state of these cells. Treatment with the test substance caused a slight but statistically significant decrease in the phagocytic index of circulating granulocytes without having any particular effect on their number, which indicates a homeostatic nature of the immunomodulating effect of the drug candidate on this population of circulating phagocytes in AD progression.

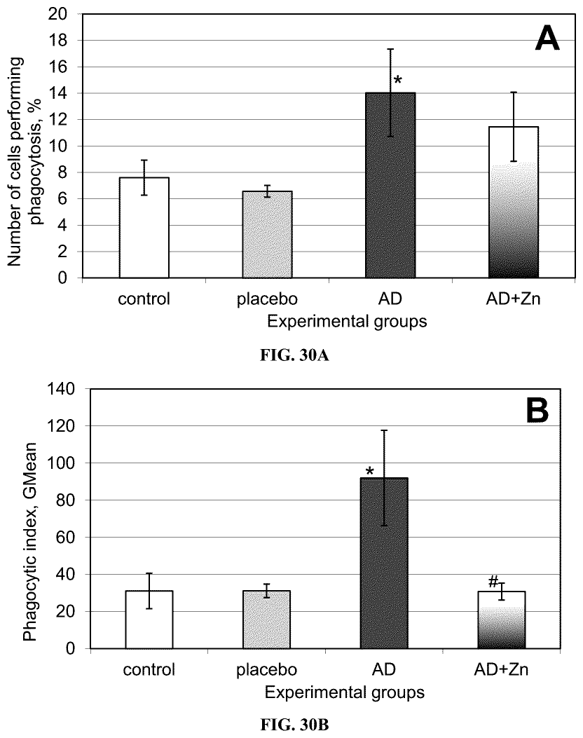

The number of monocytes performing phagocytosis in the peripheral blood of rat models of AD and their phagocytic activity were significantly higher than in the controls (FIG. 30A and FIG. 30B).

Treatment with the zinc-based preparation resulted in normalization of the analyzed parameters, which confirms our assumption about homeostatic nature of the immunomodulating effect of the test substance.

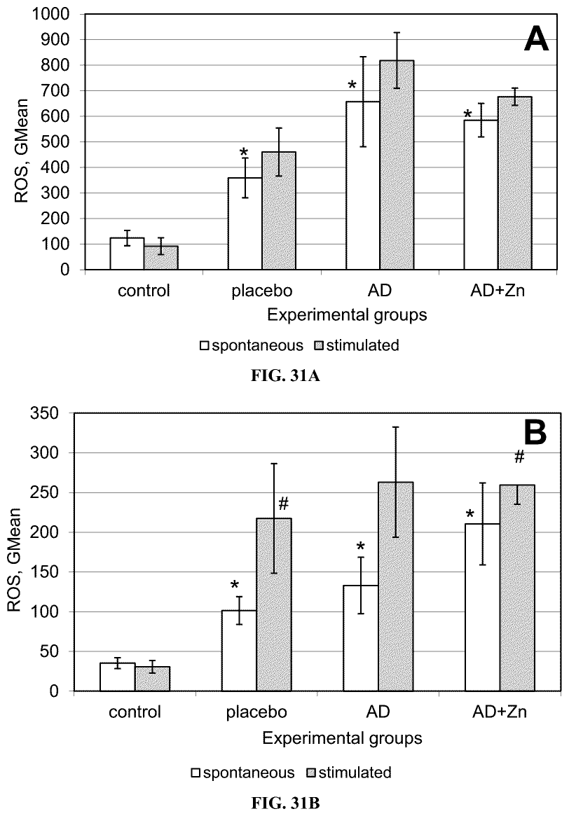

Indices of oxidative metabolism in circulating mono- and polymorphonuclear phagocytes in these rat models of AD were higher than those of intact animals and only slightly exceeded those of SO animals (FIG. 31A and FIG. 31B).

Treatment with the test substance did not cause any significant changes in the oxidative metabolism in peripheral blood phagocytes.

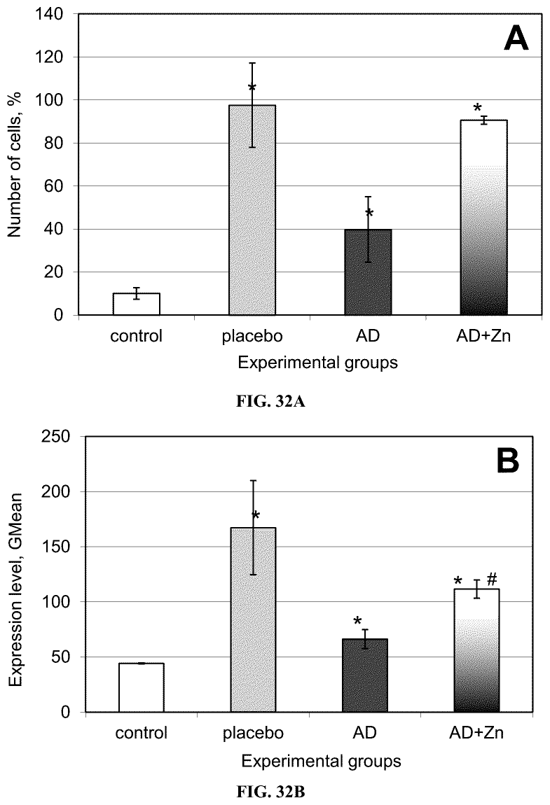

The results of assessment of the expression of phenotypic markers by peripheral blood phagocytes are difficult to interpret. A fraction of CD86+ cells and expression levels of this marker by circulating phagocytes in rat models of Aβ25-35-induced AD scarcely differed from those of intact animals and were lower than in sham-operated rats (FIG. 32A and FIG. 32B). This is most easily explained by spontaneous regression of the disease and imperfection of the AD model.

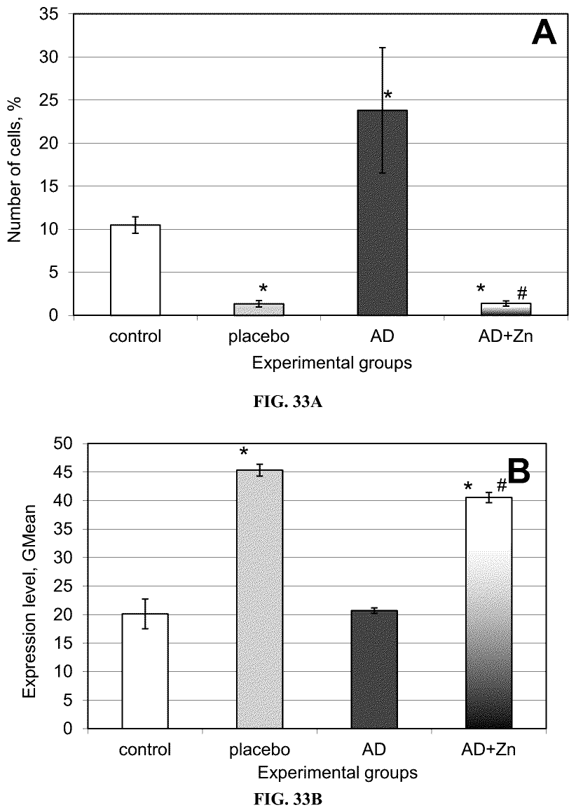

⁶⁴Zn-asp therapy caused an increase in the fraction of CD86+ cells and elevated expression of this marker, which may be evidence of enhancement of the resolution of inflammation under the action of the test substance. This assumption is also supported by the results of assessment of the CD206 expression, another phenotypic marker (FIG. 33A and FIG. 33B).

The fraction of positive cells (cells with an anti-inflammatory phenotype) was increased in Aβ25-35 injected rats. The levels of expression of this marker by blood phagocytes in AD animal models did not differ from those in the intact animals. Treatment with the test substance resulted in a sharp decrease in the fraction of cells positive for this marker but significantly increased its expression.

### Findings

Rat models of Aβ25-35-induced AD showed no significant changes in any of the selected and analyzed markers of disease progression (animal body weight, number of TH-positive neurons in the hippocampus and level of TH expression, spatial learning, short-term and long-term memories, cognitive flexibility). There was only a tendency for impairment of long-term spatial memory. ⁶⁴Zn-asp administered as monotherapy did not have any statistically significant effects on the cognitive symptoms in Aβ25-35 injected animals.

The model of AD induced by the infusion of Aβ25-35 was not characterized by a classical picture of neuroinflammation and, therefore, the protocol used in the current study does not reflect the clinical picture of Alzheimer's disease. Only the fact of possible presence of local autoimmune processes accompanying this model is noteworthy.

Therapeutic effect of the test substance on this AD model is of anti-inflammatory homeostatic nature.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

1. A method of treating Alzheimer's disease comprising administering to a subject in need thereof a therapeutically effective amount of a composition comprising Zn, wherein the composition comprises a ⁶⁴Znₑ compound or a salt thereof, wherein the ⁶⁴Znₑ compound or a salt thereof is at least 80% ⁶⁴Znₑ.

2. The method of claim 1, wherein said ⁶⁴Znₑ compound or a salt thereof is at least 95% ⁶⁴Znₑ.

3. The method of claim 1, wherein said ⁶⁴Znₑ compound or a salt thereof and said ⁶⁴Znₑ compound is at least 99% ⁶⁴Znₑ.

4. The method of claim 1, wherein the ⁶⁴Znₑ is in a form of salt selected from the group consisting of aspartate, sulfate, and citrate.

5. The method of claim 1, wherein the composition is administered by injection.

6. The method of claim 1, wherein the composition is administered orally.

7. A method of delaying the onset of Alzheimer's disease comprising administering to a subject in need thereof a prophylactically effective amount of a composition comprising Zn, wherein the composition comprises a ⁶⁴Znₑ compound or a salt thereof, wherein the 64Znₑ compound or a salt thereof is at least 80% ⁶⁴Znₑ.

8. The method of claim 7, wherein said ⁶⁴Znₑ compound or a salt thereof is at least 95% ⁶⁴Znₑ.

9. The method of claim 7, wherein said ⁶⁴Znₑ compound or a salt thereof and said 64Zne compound is at least 99% ⁶⁴Znₑ.

10. The method of claim 7, wherein the ⁶⁴Znₑ is in a form of salt selected from the group consisting of aspartate, sulfate, and citrate.

11. The method of claim 7, wherein the composition is administered by injection.

12. The method claim 7, wherein the composition is administered orally.
